SEQUENCE LISTING (126 sequences)

SEQ ID NO: 1 - DNA, 1737 bp, Artificial Sequence
  Other information  Synthetic construct
aactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacgacagcctgagcagcaacggcaacgtgaccgagagcggctgcaaggagtgcgaggagctggaggagaagaacatcaaggagttcctgcagagcttcgtgcacatcgtgcagatgttcatcaacaccagcggcggaggcggatccggaggcggaggttccggcgggggtgggagcgggcaggtgaccctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 2 - protein, 579 aa, Artificial Sequence
  Other information  Synthetic construct
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGGSGGGGSGGGGSGQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 1029 bp, Artificial Sequence
  Other information  Synthetic construct
aactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacgacagcctgagcagcaacggcaacgtgaccgagagcggctgcaaggagtgcgaggagctggaggagaagaacatcaaggagttcctgcagagcttcgtgcacatcgtgcagatgttcatcaacaccagcggcggaggcggatccggaggcggaggttccggcgggggtgggagcggggacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggctaccccttcaccttcggcggcggcaccaagctggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 4 - protein, 343 aa, Artificial Sequence
  Other information  Synthetic construct
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGGSGGGGSGGGGSGDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 1590 bp, Artificial Sequence
  Other information  Synthetic construct
atcacctgcccacctcccatgagcgtggagcacgccgacatctgggtgaagagctacagcctgtacagccgcgagcgctacatctgcaacagcggcttcaagcgcaaggccggcaccagcagcctgaccgagtgcgtgctgaacaaggccaccaacgtggcccactggaccacccccagcctgaagtgcatccgcggcggaggcggatccggaggcggaggttccggcgggggtgggagcgggcaggtgaccctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 6 - protein, 530 aa, Artificial Sequence
  Other information  Synthetic construct
ITCPPPMSVEHADIWVKSYSLYSRERYICNSGFKRKAGTSSLTECVLNKATNVAHWTTPSLKCIRGGGGSGGGGSGGGGSGQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 882 bp, Artificial Sequence
  Other information  Synthetic construct
atcacctgcccacctcccatgagcgtggagcacgccgacatctgggtgaagagctacagcctgtacagccgcgagcgctacatctgcaacagcggcttcaagcgcaaggccggcaccagcagcctgaccgagtgcgtgctgaacaaggccaccaacgtggcccactggaccacccccagcctgaagtgcatccgcggcggaggcggatccggaggcggaggttccggcgggggtgggagcggggacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggctaccccttcaccttcggcggcggcaccaagctggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 8 - protein, 294 aa, Artificial Sequence
  Other information  Synthetic construct
ITCPPPMSVEHADIWVKSYSLYSRERYICNSGFKRKAGTSSLTECVLNKATNVAHWTTPSLKCIRGGGGSGGGGSGGGGSGDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - DNA, 1737 bp, Artificial Sequence
  Other information  Synthetic construct
aactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacgacagcctgagcagcaacggcaacgtgaccgagagcggctgcaaggagtgcgaggagctggaggagaagaacatcaaggagttcctgcagagcttcgtgcacatcgtgcagatgttcatcaacaccagcggcggaggcggatccggaggcggaggttccggcgggggtgggagcggggaggtgcagctggtggagtctggaggaggcttggtccagcctggggggtccctgagactctcctgtgcagcctctgggttcaatattaaggacacttacatccactgggtccgccaggctccagggaaggggctggagtgggtcgcacgtatttatcctaccaatggttacacacgctacgcagactccgtgaagggccgattcaccatctccgcagacacttccaagaacacggcgtatcttcaaatgaacagcctgagagccgaggacacggccgtgtattactgttcgagatggggcggtgacggcttctatgccatggactactggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 10 - protein, 579 aa, Artificial Sequence
  Other information  Synthetic construct
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGGSGGGGSGGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 885 bp, Artificial Sequence
  Other information  Synthetic construct
atcacctgcccacctcccatgagcgtggagcacgccgacatctgggtgaagagctacagcctgtacagccgcgagcgctacatctgcaacagcggcttcaagcgcaaggccggcaccagcagcctgaccgagtgcgtgctgaacaaggccaccaacgtggcccactggaccacccccagcctgaagtgcatccgcggcggaggcggatccggaggcggaggttccggcgggggtgggagcggggacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaaggtggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 12 - protein, 295 aa, Artificial Sequence
  Other information  Synthetic construct
ITCPPPMSVEHADIWVKSYSLYSRERYICNSGFKRKAGTSSLTECVLNKATNVAHWTTPSLKCIRGGGGSGGGGSGGGGSGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 1590 bp, Artificial Sequence
  Other information  Synthetic construct
atcacctgcccacctcccatgagcgtggagcacgccgacatctgggtgaagagctacagcctgtacagccgcgagcgctacatctgcaacagcggcttcaagcgcaaggccggcaccagcagcctgaccgagtgcgtgctgaacaaggccaccaacgtggcccactggaccacccccagcctgaagtgcatccgcggcggaggcggatccggaggcggaggttccggcgggggtgggagcggggaggtgcagctggtggagtctggaggaggcttggtccagcctggggggtccctgagactctcctgtgcagcctctgggttcaatattaaggacacttacatccactgggtccgccaggctccagggaaggggctggagtgggtcgcacgtatttatcctaccaatggttacacacgctacgcagactccgtgaagggccgattcaccatctccgcagacacttccaagaacacggcgtatcttcaaatgaacagcctgagagccgaggacacggccgtgtattactgttcgagatggggcggtgacggcttctatgccatggactactggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 14 - protein, 530 aa, Artificial Sequence
  Other information  Synthetic construct
ITCPPPMSVEHADIWVKSYSLYSRERYICNSGFKRKAGTSSLTECVLNKATNVAHWTTPSLKCIRGGGGSGGGGSGGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 1032 bp, Artificial Sequence
  Other information  Synthetic construct
aactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacgacagcctgagcagcaacggcaacgtgaccgagagcggctgcaaggagtgcgaggagctggaggagaagaacatcaaggagttcctgcagagcttcgtgcacatcgtgcagatgttcatcaacaccagcggcggaggcggatccggaggcggaggttccggcgggggtgggagcggggacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaaggtggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 16 - protein, 344 aa, Artificial Sequence
  Other information  Synthetic construct
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGGSGGGGSGGGGSGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - DNA, 642 bp, Artificial Sequence
  Other information  Synthetic construct
gacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaaggtggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 18 - protein, 450 aa, Artificial Sequence
  Other information  Synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 642 bp, Artificial Sequence; identical to SEQ ID NO: 17
  Other information  Synthetic construct

SEQ ID NO: 20 - protein, 214 aa, Artificial Sequence
  Other information  Synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 1737 bp, Artificial Sequence
  Other information  Synthetic construct
aactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacgacagcctgagcagcaacggcaacgtgaccgagagcggctgcaaggagtgcgaggagctggaggagaagaacatcaaggagttcctgcagagcttcgtgcacatcgtgcagatgttcatcaacaccagcggcggaggcggatccggaggcggaggttccggcgggggtgggagcgggcaggtgcagctgcaggagagcggccccggcctggtgaagcccagcgagaccctgagcctgacctgcaccgtgagcggcggcagcgtgagcagcggcgactactactggacctggatccgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatagagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 22 - protein, 579 aa, Artificial Sequence
  Other information  Synthetic construct
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGGSGGGGSGGGGSGQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - DNA, 885 bp, Artificial Sequence
  Other information  Synthetic construct
atcacctgcccacctcccatgagcgtggagcacgccgacatctgggtgaagagctacagcctgtacagccgcgagcgctacatctgcaacagcggcttcaagcgcaaggccggcaccagcagcctgaccgagtgcgtgctgaacaaggccaccaacgtggcccactggaccacccccagcctgaagtgcatccgcggcggaggcggatccggaggcggaggttccggcgggggtgggagcggggacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 24 - protein, 295 aa, Artificial Sequence
  Other information  Synthetic construct
ITCPPPMSVEHADIWVKSYSLYSRERYICNSGFKRKAGTSSLTECVLNKATNVAHWTTPSLKCIRGGGGSGGGGSGGGGSGDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - DNA, 1590 bp, Artificial Sequence
  Other information  Synthetic construct
atcacctgcccacctcccatgagcgtggagcacgccgacatctgggtgaagagctacagcctgtacagccgcgagcgctacatctgcaacagcggcttcaagcgcaaggccggcaccagcagcctgaccgagtgcgtgctgaacaaggccaccaacgtggcccactggaccacccccagcctgaagtgcatccgcggcggaggcggatccggaggcggaggttccggcgggggtgggagcgggcaggtgcagctgcaggagagcggccccggcctggtgaagcccagcgagaccctgagcctgacctgcaccgtgagcggcggcagcgtgagcagcggcgactactactggacctggatccgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatagagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 26 - protein, 530 aa, Artificial Sequence
  Other information  Synthetic construct
ITCPPPMSVEHADIWVKSYSLYSRERYICNSGFKRKAGTSSLTECVLNKATNVAHWTTPSLKCIRGGGGSGGGGSGGGGSGQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - DNA, 1032 bp, Artificial Sequence
  Other information  Synthetic construct
aactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacgacagcctgagcagcaacggcaacgtgaccgagagcggctgcaaggagtgcgaggagctggaggagaagaacatcaaggagttcctgcagagcttcgtgcacatcgtgcagatgttcatcaacaccagcggcggaggcggatccggaggcggaggttccggcgggggtgggagcggggacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 28 - protein, 344 aa, Artificial Sequence
  Other information  Synthetic construct
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGGSGGGGSGGGGSGDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - DNA, 1347 bp, Artificial Sequence
  Other information  Synthetic construct
caggtgcagctgcaggagagcggccccggcctggtgaagcccagcgagaccctgagcctgacctgcaccgtgagcggcggcagcgtgagcagcggcgactactactggacctggatccgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatagagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 30 - protein, 449 aa, Artificial Sequence
  Other information  Synthetic construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - DNA, 1344 bp, Artificial Sequence
  Other information  Synthetic construct
caggtgcagctgcaggagagcggccccggcctggtgaagcccagcgagaccctgagcctgacctgcaccgtgagcggcggcagcgtgagcagcggcgactactactggacctggatccgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 32 - protein, 448 aa, Artificial Sequence
  Other information  Synthetic construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - DNA, 642 bp, Artificial Sequence
  Other information  Synthetic construct
gacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 34 - protein, 214 aa, Artificial Sequence
  Other information  Synthetic construct
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - DNA, 1341 bp, Artificial Sequence
  Other information  Synthetic construct
gaggtgcagctggtggagagcggaggtggactagtacagcctggtggcagcctacgactgagttgcgccgccagcggcttcaccttcagcgacagctggatccactgggtgcgccaggcccccggcaagggcctggagtgggtggcctggatcagcccctacggcggcagcacctactacgccgacagcgtgaagggccgcttcaccatcagcgccgacaccagcaagaacaccgcctacctgcagatgaacagcctgcgcgccgaggacaccgccgtgtactactgcgcccgccgccactggcccggcggcttcgactactggggccagggcaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 36 - protein, 447 aa, Artificial Sequence
  Other information  Synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - DNA, 642 bp, Artificial Sequence
  Other information  Synthetic construct
gacatccagatgacccagagccccagcagcctgagcgccagcgtgggcgaccgcgtgaccatcacctgccgcgccagccaggacgtgagcaccgccgtggcctggtaccagcagaagcccggcaaggcccccaagctgctgatctacagcgccagcttcctgtacagcggcgtgcccagccgcttcagcggcagcggcagcggcaccgacttcaccctgaccatcagcagcctgcagcccgaggacttcgccacctactactgccagcagtacctgtaccaccccgccaccttcggccagggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 38 - protein, 214 aa, Artificial Sequence
  Other information  Synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - DNA, 1734 bp, Artificial Sequence
  Other information  Synthetic construct
aactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacgacagcctgagcagcaacggcaacgtgaccgagagcggctgcaaggagtgcgaggagctggaggagaagaacatcaaggagttcctgcagagcttcgtgcacatcgtgcagatgttcatcaacaccagcggcggaggcggatccggaggcggaggttccggcgggggtgggagcgggcaggtgcagctgcaggagagcggccccggcctggtgaagcccagcgagaccctgagcctgacctgcaccgtgagcggcggcagcgtgagcagcggcgactactactggacctggatccgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 40 - protein, 578 aa, Artificial Sequence
  Other information  Synthetic construct
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGGSGGGGSGGGGSGQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - DNA, 1569 bp, Artificial Sequence
  Other information  Synthetic construct
atcacctgcccacctcccatgagcgtggagcacgccgacatctgggtgaagagctacagcctgtacagccgcgagcgctacatctgcaacagcggcttcaagcgcaaggccggcaccagcagcctgaccgagtgcgtgctgaacaaggccaccaacgtggcccactggaccacccccagcctgaagtgcatccgcggcggaggcggatccggaggcggaggttccggcgggggtgggagcgggcaggtgcagctggtggagagtggaggtggcgtggtacagcccggccgcagcctgcgcctggactgcaaggccagcggcatcaccttcagcaacagcggcatgcactgggtgcgccaggcccccggcaagggcctggagtgggtggccgtgatctggtacgacggcagcaagcgctactacgccgacagcgtgaagggccgcttcaccatcagccgcgacaacagcaagaacaccctgttcctgcagatgaacagcctgcgcgccgaggacaccgccgtgtactactgcgccaccaacgacgactactggggccagggcaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 42 - protein, 523 aa, Artificial Sequence
  Other information  Synthetic construct
ITCPPPMSVEHADIWVKSYSLYSRERYICNSGFKRKAGTSSLTECVLNKATNVAHWTTPSLKCIRGGGGSGGGGSGGGGSGQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - DNA, 1587 bp, Artificial Sequence
  Other information  Synthetic construct
atcacctgcccacctcccatgagcgtggagcacgccgacatctgggtgaagagctacagcctgtacagccgcgagcgctacatctgcaacagcggcttcaagcgcaaggccggcaccagcagcctgaccgagtgcgtgctgaacaaggccaccaacgtggcccactggaccacccccagcctgaagtgcatccgcggcggaggcggatccggaggcggaggttccggcgggggtgggagcgggcaggtgcagctgcaggagagcggccccggcctggtgaagcccagcgagaccctgagcctgacctgcaccgtgagcggcggcagcgtgagcagcggcgactactactggacctggatccgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 44 - protein, 529 aa, Artificial Sequence
  Other information  Synthetic construct
ITCPPPMSVEHADIWVKSYSLYSRERYICNSGFKRKAGTSSLTECVLNKATNVAHWTTPSLKCIRGGGGSGGGGSGGGGSGQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - DNA, 1731 bp, Artificial Sequence
  Other information  Synthetic construct
aactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacgacagcctgagcagcaacggcaacgtgaccgagagcggctgcaaggagtgcgaggagctggaggagaagaacatcaaggagttcctgcagagcttcgtgcacatcgtgcagatgttcatcaacaccagcggcggaggcggatccggaggcggaggttccggcgggggtgggagcggggaggtgcagctggtggagagcggaggtggactagtacagcctggtggcagcctacgactgagttgcgccgccagcggcttcaccttcagcgacagctggatccactgggtgcgccaggcccccggcaagggcctggagtgggtggcctggatcagcccctacggcggcagcacctactacgccgacagcgtgaagggccgcttcaccatcagcgccgacaccagcaagaacaccgcctacctgcagatgaacagcctgcgcgccgaggacaccgccgtgtactactgcgcccgccgccactggcccggcggcttcgactactggggccagggcaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 46 - protein, 577 aa, Artificial Sequence
  Other information  Synthetic construct
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGGSGGGGSGGGGSGEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - DNA, 885 bp, Artificial Sequence
  Other information  Synthetic construct
atcacctgcccacctcccatgagcgtggagcacgccgacatctgggtgaagagctacagcctgtacagccgcgagcgctacatctgcaacagcggcttcaagcgcaaggccggcaccagcagcctgaccgagtgcgtgctgaacaaggccaccaacgtggcccactggaccacccccagcctgaagtgcatccgcggcggaggcggatccggaggcggaggttccggcgggggtgggagcggggacatccagatgacccagagccccagcagcctgagcgccagcgtgggcgaccgcgtgaccatcacctgccgcgccagccaggacgtgagcaccgccgtggcctggtaccagcagaagcccggcaaggcccccaagctgctgatctacagcgccagcttcctgtacagcggcgtgcccagccgcttcagcggcagcggcagcggcaccgacttcaccctgaccatcagcagcctgcagcccgaggacttcgccacctactactgccagcagtacctgtaccaccccgccaccttcggccagggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 48 - protein, 295 aa, Artificial Sequence
  Other information  Synthetic construct
ITCPPPMSVEHADIWVKSYSLYSRERYICNSGFKRKAGTSSLTECVLNKATNVAHWTTPSLKCIRGGGGSGGGGSGGGGSGDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - DNA, 1584 bp, Artificial Sequence
  Other information  Synthetic construct
atcacctgcccacctcccatgagcgtggagcacgccgacatctgggtgaagagctacagcctgtacagccgcgagcgctacatctgcaacagcggcttcaagcgcaaggccggcaccagcagcctgaccgagtgcgtgctgaacaaggccaccaacgtggcccactggaccacccccagcctgaagtgcatccgcggcggaggcggatccggaggcggaggttccggcgggggtgggagcggggaggtgcagctggtggagagcggaggtggactagtacagcctggtggcagcctacgactgagttgcgccgccagcggcttcaccttcagcgacagctggatccactgggtgcgccaggcccccggcaagggcctggagtgggtggcctggatcagcccctacggcggcagcacctactacgccgacagcgtgaagggccgcttcaccatcagcgccgacaccagcaagaacaccgcctacctgcagatgaacagcctgcgcgccgaggacaccgccgtgtactactgcgcccgccgccactggcccggcggcttcgactactggggccagggcaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 50 - protein, 528 aa, Artificial Sequence
  Other information  Synthetic construct
ITCPPPMSVEHADIWVKSYSLYSRERYICNSGFKRKAGTSSLTECVLNKATNVAHWTTPSLKCIRGGGGSGGGGSGGGGSGEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - DNA, 1032 bp, Artificial Sequence
  Other information  Synthetic construct
aactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacgacagcctgagcagcaacggcaacgtgaccgagagcggctgcaaggagtgcgaggagctggaggagaagaacatcaaggagttcctgcagagcttcgtgcacatcgtgcagatgttcatcaacaccagcggcggaggcggatccggaggcggaggttccggcgggggtgggagcggggacatccagatgacccagagccccagcagcctgagcgccagcgtgggcgaccgcgtgaccatcacctgccgcgccagccaggacgtgagcaccgccgtggcctggtaccagcagaagcccggcaaggcccccaagctgctgatctacagcgccagcttcctgtacagcggcgtgcccagccgcttcagcggcagcggcagcggcaccgacttcaccctgaccatcagcagcctgcagcccgaggacttcgccacctactactgccagcagtacctgtaccaccccgccaccttcggccagggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 52 - protein, 344 aa, Artificial Sequence
  Other information  Synthetic construct
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGGSGGGGSGGGGSGDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - DNA, 1350 bp, Artificial Sequence
  Other information  Synthetic construct
caggtgaccctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 54 - protein, 450 aa, Artificial Sequence
  Other information  Synthetic construct
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - DNA, 639 bp, Artificial Sequence
  Other information  Synthetic construct
gacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggctaccccttcaccttcggcggcggcaccaagctggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 56 - protein, 213 aa, Artificial Sequence
  Other information  Synthetic construct
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - DNA, 1032 bp, Artificial Sequence
  Other information  Synthetic construct
aactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacgacagcctgagcagcaacggcaacgtgaccgagagcggctgcaaggagtgcgaggagctggaggagaagaacatcaaggagttcctgcagagcttcgtgcacatcgtgcagatgttcatcaacaccagcggcggaggcggatccggaggcggaggttccggcgggggtgggagcggggagatcgtgctgacccagagccccgccaccctgagcctgagccccggcgagcgcgccaccctgagctgccgcgccagccagagcgtgagcagctacctggcctggtaccagcagaagccaggacaggctccacgactgctaatctatgacgccagcaaccgcgccaccggcatccccgcccgcttcagcggcagcggcagcggcaccgacttcaccctgaccatcagcagcctggagcccgaggacttcgccgtgtactactgccagcagagcagcaactggccccgcaccttcggccagggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 58 - protein, 344 aa, Artificial Sequence
  Other information  Synthetic construct
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGGSGGGGSGGGGSGEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - DNA, 1740 bp, Artificial Sequence
  Other information  Synthetic construct
aactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacgacagcctgagcagcaacggcaacgtgaccgagagcggctgcaaggagtgcgaggagctggaggagaagaacatcaaggagttcctgcagagcttcgtgcacatcgtgcagatgttcatcaacaccagcggcggaggcggatccggaggcggaggttccggcgggggtgggagcggggaagtacagctgcttgagagtggaggaggtttggtacagcccggcggatccctccgcctgtcctgtgcggctagtggctttacattctcatcctatatcatgatgtgggtaagacaggccccaggaaagggcctggagtgggttagttctatctacccctcaggcgggattaccttctacgcagatactgtgaagggcaggtttaccatatcccgagacaacagtaagaataccctttaccttcaaatgaactcccttcgggccgaggacactgcggtgtactattgcgctcgcattaagcttggcaccgtgacaaccgtgaactattggggtcaaggcacgctggtgactgtctcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatagagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 60 - protein, 580 aa, Artificial Sequence
  Other information  Synthetic construct
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGGSGGGGSGGGGSGEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - DNA, 891 bp, Artificial Sequence
  Other information  Synthetic construct
atcacctgcccacctcccatgagcgtggagcacgccgacatctgggtgaagagctacagcctgtacagccgcgagcgctacatctgcaacagcggcttcaagcgcaaggccggcaccagcagcctgaccgagtgcgtgctgaacaaggccaccaacgtggcccactggaccacccccagcctgaagtgcatccgcggcggaggcggatccggaggcggaggttccggcgggggtgggagcgggcaatccgcactgactcaaccagccagcgttagcggctcccctggtcaatctatcaccatcagctgtaccgggaccagctcagacgttggcggttacaactacgtcagctggtaccagcagcacccgggtaaagctccaaagctgatgatttatgatgtgtctaatcgaccttctggtgtatctaaccgattttcaggctctaaaagtggaaatactgcttccctcacgatctcagggctgcaagccgaagacgaagccgattattattgttctagctatacatccagcagcacccgcgtgtttggaacgggaaccaaggtcacggttctgggacagcccaaagccaatcctaccgtcactctgttcccacccagtagtgaggagctgcaggcaaataaggctaccctggtctgtcttatatccgatttctatcccggggcagtcacagtcgcttggaaggcagatggctctccagtgaaggccggcgtcgaaacaactaaaccttccaagcagtctaataacaagtacgctgcttcttcttacctttcacttactcctgaacaatggaagagccacaggagttactcttgtcaggtaacccacgaggggtccactgtggagaaaaccgtcgctcccacagagtgttct

SEQ ID NO: 62 - protein, 297 aa, Artificial Sequence
  Other information  Synthetic construct
ITCPPPMSVEHADIWVKSYSLYSRERYICNSGFKRKAGTSSLTECVLNKATNVAHWTTPSLKCIRGGGGSGGGGSGGGGSGQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 63 - DNA, 1593 bp, Artificial Sequence
  Other information  Synthetic construct
atcacctgcccacctcccatgagcgtggagcacgccgacatctgggtgaagagctacagcctgtacagccgcgagcgctacatctgcaacagcggcttcaagcgcaaggccggcaccagcagcctgaccgagtgcgtgctgaacaaggccaccaacgtggcccactggaccacccccagcctgaagtgcatccgcggcggaggcggatccggaggcggaggttccggcgggggtgggagcggggaagtacagctgcttgagagtggaggaggtttggtacagcccggcggatccctccgcctgtcctgtgcggctagtggctttacattctcatcctatatcatgatgtgggtaagacaggccccaggaaagggcctggagtgggttagttctatctacccctcaggcgggattaccttctacgcagatactgtgaagggcaggtttaccatatcccgagacaacagtaagaataccctttaccttcaaatgaactcccttcgggccgaggacactgcggtgtactattgcgctcgcattaagcttggcaccgtgacaaccgtgaactattggggtcaaggcacgctggtgactgtctcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatagagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 64 - protein, 531 aa, Artificial Sequence
  Other information  Synthetic construct
ITCPPPMSVEHADIWVKSYSLYSRERYICNSGFKRKAGTSSLTECVLNKATNVAHWTTPSLKCIRGGGGSGGGGSGGGGSGEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - DNA, 1038 bp, Artificial Sequence
  Other information  Synthetic construct
aactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacgacagcctgagcagcaacggcaacgtgaccgagagcggctgcaaggagtgcgaggagctggaggagaagaacatcaaggagttcctgcagagcttcgtgcacatcgtgcagatgttcatcaacaccagcggcggaggcggatccggaggcggaggttccggcgggggtgggagcgggcaatccgcactgactcaaccagccagcgttagcggctcccctggtcaatctatcaccatcagctgtaccgggaccagctcagacgttggcggttacaactacgtcagctggtaccagcagcacccgggtaaagctccaaagctgatgatttatgatgtgtctaatcgaccttctggtgtatctaaccgattttcaggctctaaaagtggaaatactgcttccctcacgatctcagggctgcaagccgaagacgaagccgattattattgttctagctatacatccagcagcacccgcgtgtttggaacgggaaccaaggtcacggttctgggacagcccaaagccaatcctaccgtcactctgttcccacccagtagtgaggagctgcaggcaaataaggctaccctggtctgtcttatatccgatttctatcccggggcagtcacagtcgcttggaaggcagatggctctccagtgaaggccggcgtcgaaacaactaaaccttccaagcagtctaataacaagtacgctgcttcttcttacctttcacttactcctgaacaatggaagagccacaggagttactcttgtcaggtaacccacgaggggtccactgtggagaaaaccgtcgctcccacagagtgttct

SEQ ID NO: 66 - protein, 346 aa, Artificial Sequence
  Other information  Synthetic construct
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGGSGGGGSGGGGSGQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 67 - DNA, 1740 bp, Artificial Sequence
  Other information  Synthetic construct
aactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacgacagcctgagcagcaacggcaacgtgaccgagagcggctgcaaggagtgcgaggagctggaggagaagaacatcaaggagttcctgcagagcttcgtgcacatcgtgcagatgttcatcaacaccagcggcggaggcggatccggaggcggaggttccggcgggggtgggagcgggcaggtgaccctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatagagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 68 - protein, 563 aa, Artificial Sequence
  Other information  Synthetic construct
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGGSGGGGSGGGGSGQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHE

SEQ ID NO: 69 - DNA, 1593 bp, Artificial Sequence
  Other information  Synthetic construct
atcacctgcccacctcccatgagcgtggagcacgccgacatctgggtgaagagctacagcctgtacagccgcgagcgctacatctgcaacagcggcttcaagcgcaaggccggcaccagcagcctgaccgagtgcgtgctgaacaaggccaccaacgtggcccactggaccacccccagcctgaagtgcatccgcggcggaggcggatccggaggcggaggttccggcgggggtgggagcgggcaggtgaccctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatagagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 70 - protein, 531 aa, Artificial Sequence
  Other information  Synthetic construct
ITCPPPMSVEHADIWVKSYSLYSRERYICNSGFKRKAGTSSLTECVLNKATNVAHWTTPSLKCIRGGGGSGGGGSGGGGSGQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - DNA, 342 bp, Artificial Sequence
  Other information  Synthetic construct
aactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacgacagcctgagcagcaacggcaacgtgaccgagagcggctgcaaggagtgcgaggagctggaggagaagaacatcaaggagttcctgcagagcttcgtgcacatcgtgcagatgttcatcaacaccagc

SEQ ID NO: 72 - protein, 114 aa, Artificial Sequence
  Other information  Synthetic construct
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 73 - DNA, 195 bp, Artificial Sequence
  Other information  Synthetic construct
atcacctgcccacctcccatgagcgtggagcacgccgacatctgggtgaagagctacagcctgtacagccgcgagcgctacatctgcaacagcggcttcaagcgcaaggccggcaccagcagcctgaccgagtgcgtgctgaacaaggccaccaacgtggcccactggaccacccccagcctgaagtgcatccgc

SEQ ID NO: 74 - protein, 65 aa, Artificial Sequence
  Other information  Synthetic construct
ITCPPPMSVEHADIWVKSYSLYSRERYICNSGFKRKAGTSSLTECVLNKATNVAHWTTPSLKCIR

SEQ ID NO: 75 - DNA, 1326 bp, Artificial Sequence
  Other information  Synthetic construct
caggtgcagctggtggagagtggaggtggcgtggtacagcccggccgcagcctgcgcctggactgcaaggccagcggcatcaccttcagcaacagcggcatgcactgggtgcgccaggcccccggcaagggcctggagtgggtggccgtgatctggtacgacggcagcaagcgctactacgccgacagcgtgaagggccgcttcaccatcagccgcgacaacagcaagaacaccctgttcctgcagatgaacagcctgcgcgccgaggacaccgccgtgtactactgcgccaccaacgacgactactggggccagggcaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 76 - protein, 442 aa, Artificial Sequence
  Other information  Synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - DNA, 642 bp, Artificial Sequence
  Other information  Synthetic construct
gagatcgtgctgacccagagccccgccaccctgagcctgagccccggcgagcgcgccaccctgagctgccgcgccagccagagcgtgagcagctacctggcctggtaccagcagaagccaggacaggctccacgactgctaatctatgacgccagcaaccgcgccaccggcatccccgcccgcttcagcggcagcggcagcggcaccgacttcaccctgaccatcagcagcctggagcccgaggacttcgccgtgtactactgccagcagagcagcaactggccccgcaccttcggccagggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 78 - protein, 214 aa, Artificial Sequence
  Other information  Synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - DNA, 1350 bp, Artificial Sequence
  Other information  Synthetic construct
gaagtacagctgcttgagagtggaggaggtttggtacagcccggcggatccctccgcctgtcctgtgcggctagtggctttacattctcatcctatatcatgatgtgggtaagacaggccccaggaaagggcctggagtgggttagttctatctacccctcaggcgggattaccttctacgcagatactgtgaagggcaggtttaccatatcccgagacaacagtaagaataccctttaccttcaaatgaactcccttcgggccgaggacactgcggtgtactattgcgctcgcattaagcttggcaccgtgacaaccgtgaactattggggtcaaggcacgctggtgactgtctcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatagagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 80 - protein, 450 aa, Artificial Sequence
  Other information  Synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - DNA, 648 bp, Artificial Sequence
  Other information  Synthetic construct
caatccgcactgactcaaccagccagcgttagcggctcccctggtcaatctatcaccatcagctgtaccgggaccagctcagacgttggcggttacaactacgtcagctggtaccagcagcacccgggtaaagctccaaagctgatgatttatgatgtgtctaatcgaccttctggtgtatctaaccgattttcaggctctaaaagtggaaatactgcttccctcacgatctcagggctgcaagccgaagacgaagccgattattattgttctagctatacatccagcagcacccgcgtgtttggaacgggaaccaaggtcacggttctgggacagcccaaagccaatcctaccgtcactctgttcccacccagtagtgaggagctgcaggcaaataaggctaccctggtctgtcttatatccgatttctatcccggggcagtcacagtcgcttggaaggcagatggctctccagtgaaggccggcgtcgaaacaactaaaccttccaagcagtctaataacaagtacgctgcttcttcttacctttcacttactcctgaacaatggaagagccacaggagttactcttgtcaggtaacccacgaggggtccactgtggagaaaaccgtcgctcccacagagtgttct

SEQ ID NO: 82 - protein, 216 aa, Artificial Sequence
  Other information  Synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 83 - DNA, 1716 bp, Artificial Sequence
  Other information  Synthetic construct
aactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacgacagcctgagcagcaacggcaacgtgaccgagagcggctgcaaggagtgcgaggagctggaggagaagaacatcaaggagttcctgcagagcttcgtgcacatcgtgcagatgttcatcaacaccagcggcggaggcggatccggaggcggaggttccggcgggggtgggagcgggcaggtgcagctggtggagagtggaggtggcgtggtacagcccggccgcagcctgcgcctggactgcaaggccagcggcatcaccttcagcaacagcggcatgcactgggtgcgccaggcccccggcaagggcctggagtgggtggccgtgatctggtacgacggcagcaagcgctactacgccgacagcgtgaagggccgcttcaccatcagccgcgacaacagcaagaacaccctgttcctgcagatgaacagcctgcgcgccgaggacaccgccgtgtactactgcgccaccaacgacgactactggggccagggcaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 84 - protein, 572 aa, Artificial Sequence
  Other information  Synthetic construct
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGGSGGGGSGGGGSGQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - DNA, 885 bp, Artificial Sequence
  Other information  Synthetic construct
atcacctgcccacctcccatgagcgtggagcacgccgacatctgggtgaagagctacagcctgtacagccgcgagcgctacatctgcaacagcggcttcaagcgcaaggccggcaccagcagcctgaccgagtgcgtgctgaacaaggccaccaacgtggcccactggaccacccccagcctgaagtgcatccgcggcggaggcggatccggaggcggaggttccggcgggggtgggagcggggagatcgtgctgacccagagccccgccaccctgagcctgagccccggcgagcgcgccaccctgagctgccgcgccagccagagcgtgagcagctacctggcctggtaccagcagaagccaggacaggctccacgactgctaatctatgacgccagcaaccgcgccaccggcatccccgcccgcttcagcggcagcggcagcggcaccgacttcaccctgaccatcagcagcctggagcccgaggacttcgccgtgtactactgccagcagagcagcaactggccccgcaccttcggccagggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 86 - protein, 295 aa, Artificial Sequence
  Other information  Synthetic construct
ITCPPPMSVEHADIWVKSYSLYSRERYICNSGFKRKAGTSSLTECVLNKATNVAHWTTPSLKCIRGGGGSGGGGSGGGGSGEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - DNA, 711 bp, Artificial Sequence
  Other information  Synthetic construct
atcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccaccta

SEQ ID NO: 88 - protein, 237 aa, Artificial Sequence
  Other information  Synthetic construct
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 89 - DNA, 1737 bp, Artificial Sequence
  Other information  Synthetic construct
aactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagcggcggaggcggatccggtcctctgggagtacgaggcgggggtgggagcgggcaggtgcagctgcaggagagcggccccggcctggtgaagcccagcgagaccctgagcctgacctgcaccgtgagcggcggcagcgtgagcagcggcgactactactggacctggattcgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 90 - protein, 579 aa, Artificial Sequence
  Other information  Synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGPLGVRGGGGSGQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - DNA, 1719 bp, Artificial Sequence
  Other information  Synthetic construct
aactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagcggcggaggcggatccggtcctctgggagtacgacaggtgcagctgcaggagagcggccccggcctggtgaagcccagcgagaccctgagcctgacctgcaccgtgagcggcggcagcgtgagcagcggcgactactactggacctggattcgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 92 - protein, 573 aa, Artificial Sequence
  Other information  Synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGPLGVRQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - DNA, 1737 bp, Artificial Sequence
  Other information  Synthetic construct
caggtgcagctgcaggagagcggccccggcctggtgaagcccagcgagaccctgagcctgacctgcaccgtgagcggcggcagcgtgagcagcggcgactactactggacctggattcgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggcggaggcggatccggtcctctgggagtacgaggcgggggtgggagcgggaactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagc

SEQ ID NO: 94 - protein, 579 aa, Artificial Sequence
  Other information  Synthetic construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGPLGVRGGGGSGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 95 - DNA, 1722 bp, Artificial Sequence
  Other information  Synthetic construct
caggtgcagctgcaggagagcggccccggcctggtgaagcccagcgagaccctgagcctgacctgcaccgtgagcggcggcagcgtgagcagcggcgactactactggacctggattcgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtcctctgggagtacgaggcgggggtgggagcgggaactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagc

SEQ ID NO: 96 - protein, 574 aa, Artificial Sequence
  Other information  Synthetic construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGPLGVRGGGGSGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 97 - DNA, 1740 bp, Artificial Sequence
  Other information  Synthetic construct
aactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagcggcggaggcggatccggtcctctgggagtacgaggcgggggtgggagcggggaggtgcagctggtggagtctggaggaggcttggtccagcctggggggtccctgagactctcctgtgcagcctctgggttcaatattaaggacacttacatccactgggtccgccaggctccagggaaggggctggagtgggtcgcacgtatttatcctaccaatggttacacacgctacgcagactccgtgaagggccgattcaccatctccgcagacacttccaagaacacggcgtatcttcaaatgaacagcctgagagccgaggacacggccgtgtattactgttcgagatggggcggtgacggcttctatgccatggactactggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 98 - protein, 580 aa, Artificial Sequence
  Other information  Synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGPLGVRGGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - DNA, 1722 bp, Artificial Sequence
  Other information  Synthetic construct
aactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagcggcggaggcggatccggtcctctgggagtacgagaggtgcagctggtggagtctggaggaggcttggtccagcctggggggtccctgagactctcctgtgcagcctctgggttcaatattaaggacacttacatccactgggtccgccaggctccagggaaggggctggagtgggtcgcacgtatttatcctaccaatggttacacacgctacgcagactccgtgaagggccgattcaccatctccgcagacacttccaagaacacggcgtatcttcaaatgaacagcctgagagccgaggacacggccgtgtattactgttcgagatggggcggtgacggcttctatgccatggactactggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 100 - protein, 574 aa, Artificial Sequence
  Other information  Synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGPLGVREVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - DNA, 1035 bp, Artificial Sequence
  Other information  Synthetic construct
aactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagcggcggaggcggatccggtcctctgggagtacgaggcgggggtgggagcggggacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaaggttgagatcaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 102 - protein, 345 aa, Artificial Sequence
  Other information  Synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGPLGVRGGGGSGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - DNA, 1017 bp, Artificial Sequence
  Other information  Synthetic construct
aactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagcggcggaggcggatccggtcctctgggagtacgagacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaaggttgagatcaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 104 - protein, 339 aa, Artificial Sequence
  Other information  Synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGPLGVRDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - DNA, 1737 bp, Artificial Sequence
  Other information  Synthetic construct
aactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagcggcggaggcggatccggaggcggaggttccggcgggggtgggagcggggaggtgcagctggtggagtctggaggaggcttggtccagcctggggggtccctgagactctcctgtgcagcctctgggttcaatattaaggacacttacatccactgggtccgccaggctccagggaaggggctggagtgggtcgcacgtatttatcctaccaatggttacacacgctacgcagactccgtgaagggccgattcaccatctccgcagacacttccaagaacacggcgtatcttcaaatgaacagcctgagagccgaggacacggccgtgtattactgttcgagatggggcggtgacggcttctatgccatggactactggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 106 - protein, 579 aa, Artificial Sequence
  Other information  Synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - DNA, 1032 bp, Artificial Sequence
  Other information  Synthetic construct
aactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagcggcggaggcggatccggaggcggaggttccggcgggggtgggagcggggacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaaggttgagatcaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 108 - protein, 344 aa, Artificial Sequence
  Other information  Synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - DNA, 1347 bp, Artificial Sequence
  Other information  Synthetic construct
gaggtgcagctggtggagtctggaggaggcttggtccagcctggggggtccctgagactctcctgtgcagcctctgggttcaatattaaggacacttacatccactgggtccgccaggctccagggaaggggctggagtgggtcgcacgtatttatcctaccaatggttacacacgctacgcagactccgtgaagggccgattcaccatctccgcagacacttccaagaacacggcgtatcttcaaatgaacagcctgagagccgaggacacggccgtgtattactgttcgagatggggcggtgacggcttctatgccatggactactggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 110 - protein, 449 aa, Artificial Sequence
  Other information  Synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - DNA, 342 bp, Artificial Sequence
  Other information  Synthetic construct
aactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagc

SEQ ID NO: 112 - protein, 114 aa, Artificial Sequence
  Other information  Synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 113 - protein, 578 aa, Artificial Sequence
  Other information  Synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSGQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 578 aa, Artificial Sequence
  Other information  Synthetic construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 115 - protein, 344 aa, Artificial Sequence
  Other information  Synthetic construct
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 116 - protein, 308 aa, Artificial Sequence
  Other information  Synthetic construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSGITCPPPMSVEHADIWVKSYSLYSRERYICNSGFKRKAGTSSLTECVLNKATNVAHWTTPSLKCIR

SEQ ID NO: 117 - protein, 357 aa, Artificial Sequence
  Other information  Synthetic construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSGNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 118 - protein, 295 aa, Artificial Sequence
  Other information  Synthetic construct
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGITCPPPMSVEHADIWVKSYSLYSRERYICNSGFKRKAGTSSLTECVLNKATNVAHWTTPSLKCIR

SEQ ID NO: 119 - protein, 580 aa, Artificial Sequence
  Other information  Synthetic construct
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGGSGGGGSGGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 531 aa, Artificial Sequence
  Other information  Synthetic construct
ITCPPPMSVEHADIWVKSYSLYSRERYICNSGFKRKAGTSSLTECVLNKATNVAHWTTPSLKCIRGGGGSGGGGSGGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 16 aa, Artificial Sequence
  linker
GGGGSGGGGSGGGGSG

SEQ ID NO: 122 - protein, 9 aa, Artificial Sequence
  linker
GSPLGVRGS

SEQ ID NO: 123 - protein, 7 aa, Artificial Sequence
  linker
GSPLGVR

SEQ ID NO: 124 - protein, 5 aa, Artificial Sequence
  linker
PLGVR

SEQ ID NO: 125 - protein, 17 aa, Artificial Sequence
  linker
GGGGSGPLGVRGGGGSG

SEQ ID NO: 126 - protein, 11 aa, Artificial Sequence
  linker
GGGGSGPLGVR

(19) United States
(12) Patent Application Publication
(10) Pub. No.: US 2022/0289838 A1
(43) Pub. Date: Sep. 15, 2022

(54) Immunocytokine, Preparation for the Same, and Uses Thereof

(71) Applicant: NANTONG YICHEN BIOPHARMA. CO. LTD., Nantong (CN)

(72) Inventors: Feng WANG, Shanghai (CN); Huayang ZHENG, Shanghai (CN); Yuhan ZHANG, Shanghai (CN)

(21) Appl. No.: 17/636,352

(22) PCT Filed: Aug. 19, 2020
(86) PCT No.: PCT/CN2020/109986

§ 371 Date: Feb. 17, 2022

(30) Foreign Application Priority Data
Aug. 19, 2019 (CN) 201910764762.1

(51) Int. Cl. C07K 16/28; C07K 14/54; C07K 14/715; C12N 15/63; A61P 35/00

(52) U.S. Cl. CPC C07K 16/28; C07K 14/5443; C07K 14/7155; C12N 15/63; A61P 35/00

(57) ABSTRACT

The present disclosure provides an immunocytokine with IL-15 and IL-15Ra fused to an antibody targeting a tumor cell surface antigen, which can effectively and specifically target the complex of IL-15 and IL-15Ra to tumor microenvironment, and activate relevant immune cells within or in the vicinity of a tumor, thereby achieving the goal of tumor-specific killing, and at the same time, preventing immunotoxicity induced by the systemic hyperactivation of NK cells.

### CROSS-REFERENCES TO RELATED APPLICATION

The present application is a National Stage of International Patent Application No: PCT/CN2020/109986 filed on Aug. 19, 2020, which claims the benefit of the priority of the Chinese patent application with the application No. 201910764762.1, filed to the China National Intellectual Property Administration on Aug. 19, 2019, the entire content of which is incorporated in this application by reference.

### SEQUENCE LISTING

The present application contains a Sequence Listing which has been submitted electronically in ASCII format and is hereby incorporated by reference in its entirety. Said ASCII copy is 336,896 bytes in size, and contains 6 new sequences from SEQ ID NO:121 to SEQ ID NO:126 described in Specification of this file, but not numbered. The original sequences of SEQ ID NO: 1 to SEQ ID NO:120 are identical to the sequence listing filed in the corresponding international application No. PCT/CN2020/109986 filed on Aug. 19, 2020.

### TECHNICAL FIELD

The present disclosure belongs to the field of bio-pharmaceuticals, and particularly relates to an immunocytokine in which IL-15 and IL-15Ra are fused to antibody or an immunocytokine in which IL-15 is fused to antibody, and preparation for the same, and uses thereof.

### BACKGROUND

Over the past few years, immunotherapy has been used to treat various type of human tumors. Immunotherapy attempts to harness the power of the immune system to attack or kill malignant tumor cells while leaving healthy tissues intact. Immune system has the ability to identify and eliminate malignant tumor cells, however, tumors have developed a variety of mechanisms to escape immune surveillance. The challenge of immunotherapy is to develop strategies capable of effectively and safely augmenting anti-tumor responses of the body. the tumor immunotherapy strategies currently developed mainly include cytokine therapy, adoptive cell transfer therapy, cancer vaccines, and monoclonal antibody. The most promising of these strategies are those stimulating or activating the tumor-specific responses of T cells or NK that exerting the killing effect on tumors [The Potential and Promise of IL-15 in Immuno-Oncogenic Therapies].

IL-15 is a cytokine with a structure similar to IL-2, which can effectively regulate the development, proliferation and activation of effector, natural killer cells (N K), and memory CD8+ T cells. IL-15Ra is a transmembrane protein with high affinity to IL-15. After binding to IL-15, IL-15Ra is trans-presented to the surface of NK cells or T cells expressing IL2R beta and gamma chains, promoting proliferation and activation of these cells, and enhancing tumor-killing activity. Moreover, different from IL-2, IL-15 will not lead to the activation of Treg cells, and is currently undergoing extensive preclinical and clinical study as a tumor immunotherapy agent.

The complex (IL-15 superagonist) formed by IL-15 and IL-15Ra further accelerates the study of IL-15-based tumor immunotherapy. Compared with the monomer IL-15, IL-15 superagonists have prominent advantages, such as higher blood circulation concentration, longer in vivo half-life and enhanced ability to stimulate NK and CD8T cells. Currently, there are two main types of IL-15 superagonists currently under development: RLI (e.g., Cytune's CYP0150) formed by the fusion of IL-15 with IL-15Ra via a linker, and intramolecular complexes (e.g., NVS NIZ985, Altor ALT-803, and Xencor XmAb24306) formed by IL-15 and IL-15Ra. CYP0150, is easily cleared in vivo, and has a short half-life due to the absence of Fc, requiring frequent administration. The structure of NIZ985 and ALT-803, in which IL-15 and IL-15Ra form complex through intermolecular interaction, has unstable structure, leading to complicated preparation process. XmAb24306 with IL-15 and IL-15Ra fused to N-terminus of Fc, respectively, has a longer in vivo half-life due to the stable structure in which IL-15 and IL-15Ra fused to N-terminus of Fc, respectively, forming intramolecular complex. A large number of preclinical studies have indicated that the physiological and anti-tumor activity of IL-15 superagonists are greatly enhanced when comparing to that of natural IL-15. Some studies have indicated that immunotherapy such as Nivolumab (anti-PD-1 monoclonal antibody) and Rituximab (anti-CD20 monoclonal antibody) can effectively enhance the anti-tumor effect of ALT-803 in patients with progressive tumor. Moreover, the systemic administration of the IL-15 superagonist can bias the expansion of pro-inflammatory (CD11b_{high}CD27_{high}) NK cell subsets in lymphoid organs, and mass secretion of proinflammatory factors such as IFNγ, leading to immune-related adverse effects such as hypothermia, weight loss, liver damage.

The inventor finds that the immunocytokine, in which IL-15 and IL-15Ra are fused with antibody targeting tumor antigen, respectively, may specifically target tumor microenvironment. In tumor microenvironment, the complex formed by IL-15/IL15Ra in the immunocytokine may activate immune cells within or in the vicinity of a tumor, thereby exerting the specific killing effect of T cells or NK cells on the tumor while preventing immune-related adverse effects induced by the systemic hyperactivation of T cells or NK cells.

### SUMMARY

The inventor finds that the immunocytokine, in which IL-15 and IL-15Ra are fused to an antibody targeting therapy-related cell surface antigen, respectively, can effectively and specifically target tumor microenvironment, and activate immune cells within or in the vicinity of a tumor, exerting specific killing effects on tumor while preventing immune-related adverse effects induced by the systemic hyperactivation of NK cells or T cells.

One aspect of the present disclosure provides an immunocytokine, which includes:

(A) interleukin-15 (IL-15);

(B) interleukin-15 receptor-a subunit (IL-15Ra);

(C) an antibody targeting a therapy-related cell surface antigen;

wherein, IL-15 is fused to the N-terminal of the heavy chain variable region of the antibody directly or via a linker, and IL-15Ra is fused to N-terminal of the light chain variable region of the antibody directly or via a liner; or, the IL-15 is fused to N-terminal of the light chain variable region of the antibody, and IL-15Ra is fused to N-terminal of the heavy chain variable region of the antibody directly or via a linker; or, the IL-15 is fused to C-terminal of the light chain constant region (CL) of the antibody directly or via a linker, and IL-15Ra is fused to C-terminal of the heavy chain constant region (CH1) of the antibody directly or via a linker; or the IL-15 is fused to C-terminal of the heavy chain constant region (CH1) of the antibody directly or via a linker, and IL-15Ra is, fused to a C-terminal of the light chain constant region (CL) of the antibody directly or via a linker. In some embodiments, the linker is a cleavable or non-cleavable. In some embodiments, the linker is independently selected from, but not limited to GGGGSGGGGSGGGGSG (SEQ ID NO:121), GSPLGVRGS (SEQ ID NO:122), GSPLGVR (SEQ ID NO:123), PLGVR (SEQ ID NO:124), GGGGSGPLGVRGGGGSG (SEQ ID NO:125) or GGGGSGPLGVR (SEQ ID NO:126) and the like.

In some embodiments, IL-15 is a mammalian IL-15, preferably, a primate IL-15, more preferably, a human IL-15. In some embodiments, IL-15 is a wild-type IL-15 or IL-15 derivative. In some embodiments, IL-15 has a nucleotide sequence as shown in SEQ ID NO: 71 or an amino acid sequence as shown in SEQ ID NO: 72; in some embodiments, IL-15 has a nucleotide sequence as shown in SEQ ID NO: 111 or an amino acid sequence as shown in SEQ ID NO: 112. In some other embodiments, IL-15 has an amino acid sequence which has a percentage of identity of 85%-100% with SEQ ID NO: 72 or SEQ ID NO: 112.

In some embodiments, the IL-15Ra is IL-15Ra. In some embodiments, the IL-15Ra is an IL-15Ra Sushi domain. In some embodiments, IL-15Ra is a variant or derivative of IL-15Ra Sushi domain. In some other embodiments, IL-15Ra has a nucleotide sequence as shown in SEQ ID NO: 73 or an amino acid sequence as shown in SEQ ID NO: 74. In some other embodiments, IL-15Ra has a nucleotide sequence as shown in SEQ ID NO: 87 or an amino acid sequence as shown in SEQ ID NO: 88. In some other embodiments, IL-15Ra has an amino acid sequence which has a percentage of identity of 85%-100% with SEQ ID NO: 74 or SEQ ID NO: 88.

In some other embodiments, the therapy-related cell surface antigen is a CD antigen. In some other embodiments, the CD antigen is selected from but not limited to, CD19, CD20, CD47, CD40, CD73, CD33, CD38, CD123, CD30, CD3, CD22, CD25, CD133, CD27, CD39, CD138, CD46, CD56, CD70, CD32, CD11b, CD135, CD171, CD174, CD147, CD155, CD16, CD162, CD16a, CD200, CD21, CD28, CD44, CD52, CD54, CD7, CD80, CD88, CD13, CD130, CD150, CD160, CD200R1, CD267, CD29, CD3E, CD4, CD51 or CD8, or the like.

In some other embodiments, the therapy-related cell surface antigen is an immune checkpoint protein. In some other embodiments, the immune checkpoint protein is selected from but not limited to PD-1, PD-L1, CTLA-4, LAG-3, OX40, CD28, CD40, CD47, CD70, CD80, CD122, GTIR, A2AR, B7-H3 (CD276), B7-H4, IDO, KIR, Tim-3 or 4-1 BB(CD137), or the like. In some other embodiments, the immune checkpoint protein is PD-1. In some other embodiments, the antibody targeting the immune checkpoint protein PD-1 has sequences of HCDR1, HCDR2 and HCDR3 contained in heavy chain as shown in SEQ ID NO: 76, and sequences of LCDR1, LCDR2 and LCDR3 contained in light chain as shown in SEQ ID NO: 78; or has a VH sequence contained in the heavy chain as shown in SEQ ID NO: 76, and a VL sequence contained in the light chain as shown in SEQ ID NO: 78. In some embodiments, the antibody targeting the immune checkpoint protein PD-1 has a heavy chain nucleotide sequence as shown in SEQ ID NO: 75 and a light chain nucleotide sequence as shown in SEQ ID NO: 77; or has a heavy chain amino acid sequence as shown in SEQ ID NO: 76 and a light chain amino acid sequence as shown in SEQ ID NO: 78.

In some other embodiments, the immune checkpoint protein is PD-L1. In some other embodiments, antibody targeting the immune checkpoint protein PD-L1 has sequences of HCDR1, HCDR2 and HCDR3 contained in heavy chain as shown in SEQ ID NO: 36, and sequences of LCDR1, LCDR2 and LCDR3 contained in light chain as shown in SEQ ID NO: 38; or has sequences of HCDR1, HCDR2 and HCDR3 contained in heavy chain as shown in SEQ ID NO: 80, and sequences of LCDR1, LCDR2 and LCDR3 contained in light chain as shown in SEQ ID NO: 82. In some other embodiments, the antibody targeting the immune checkpoint protein PD-L1 has a VH sequence contained in an amino acid sequence as shown in SEQ ID NO: 36, and a VL sequence contained in an amino acid sequence as shown in SEQ ID NO: 38; or has a VH sequence contained in the heavy chain as shown in SEQ ID NO: 80, and a VL sequence contained in the light chain as shown in SEQ ID NO: 82. In some embodiments, the antibody targeting the immune checkpoint protein PD-L1 has a heavy chain nucleotide sequence as shown in SEQ ID NO: 35 and a light chain nucleotide sequence as shown in SEQ ID NO: 37; or has a heavy chain amino acid sequence as shown in SEQ ID NO: 36 and a light chain amino acid sequence as shown in SEQ ID NO: 38. In some embodiments, the antibody targeting the immune checkpoint protein PD-L1 has a heavy chain nucleotide sequence as shown in SEQ ID NO: 79 and a light chain nucleotide sequence as shown in SEQ ID NO: 81; or has a heavy chain amino acid sequence as shown in SEQ ID NO: 80 and a light chain amino acid sequence as shown in SEQ ID NO: 82.

In some other embodiments, the therapy-related cell surface antigen is a tumor surface antigen. In some other embodiments, the tumor surface antigen is selected from but not limited to EGFR, HER2, HER3, HER4, NY-ESO-1, GPC-3, CLL-1, BCMA, GD2, EpCAM, a chemical chemokine receptor family (CCR1, CCR2, CCR3, CCR4, CCR5, CCR6, CCR7, CCR8, CCR9, CCR10, CCL27, CCL28, CX3CR1, CXCR1, CXCR2, CXCR3, CXCR4, CXCR5, CXCR6), a mucin family (MUC1, MUC2, MUC3A, MUC3B, MUC4, MUC5AC, MUC5B, MUC6, MUC7, MUC8, MUC12, MUC13, MUC15, MUC16, MUC17, MUC19, MUC20), PSMA, CEA, HDAC6, Mesothelin, TERT, TLR, TLR9, TLR4, CD33, GITR, Survivin, CD123, TIGIT, fibroblast growth factor receptors (FGFR), vascular endothelial growth factors (FLT1, KDR/Flk-1, and VEGFR-3), hepatocyte growth factor receptors (HGFR), nerve growth factor receptors (NGFR), insulin-like growth factor receptors (IGFR), platelet-derived growth factor receptors (PDGFR), and hormone receptors (melanocortin 1 receptors (MC1R, MSHR), and the like.

In some other embodiments, the tumor surface antigen is EGFR. In some other embodiments, the antibody targeting EGFR has sequences of HCDR1, HCDR2 and HCDR3 contained in heavy chain as shown in SEQ ID NO: 32, and sequences of LCDR1, LCDR2 and LCDR3 contained in light chain as shown in SEQ ID NO: 34; or has a VH sequence contained in the heavy chain as shown in SEQ ID NO: 32, and a VL sequence contained in the light chain as shown in SEQ ID NO: 34. In some embodiments, the antibody targeting the tumor surface antigen EGFR has a heavy chain nucleotide sequence as shown in SEQ ID NO: 29 and a light chain nucleotide sequence as shown in SEQ ID NO: 33; or has a heavy chain amino acid sequence as shown in SEQ ID NO: 30 and a light chain amino acid sequence as shown in SEQ ID NO: 34. In some embodiments, the antibody targeting the tumor surface antigen EGFR has a heavy chain nucleotide sequence as shown in SEQ ID NO: 31 and a light chain nucleotide sequence as shown in SEQ ID NO: 33; or has a heavy chain amino acid sequence as shown in SEQ ID NO: 32 and a light chain amino acid sequence as shown in SEQ ID NO: 34.

In some other embodiments, the tumor surface antigen is HER2. In some other embodiments, the antibody targeting HER2 has sequences of HCDR1, HCDR2 and HCDR3 contained in heavy chain amino as shown in SEQ ID NO: 18, and sequences of LCDR1, LCDR2 and LCDR3 contained in light chain as shown in SEQ ID NO: 20; or the antibody targeting HER2 has a VH sequence contained in the heavy chain as shown in SEQ ID NO: 18, and a VL sequence contained in the light chain as shown in SEQ ID NO: 20. In some embodiments, the antibody targeting HER2 has a heavy chain nucleotide sequence as shown in SEQ ID NO: 17 and a light chain nucleotide sequence as shown in SEQ ID NO: 19; or has a heavy chain amino acid sequence as shown in SEQ ID NO: 18 and a light chain amino acid sequence as shown in SEQ ID NO: 20.

In some other embodiments, the cell surface antigen is selected from a virus-associated antigen. In some other embodiments, the virus-associated antigen is selected from but not limited to RSV F, HPV E6, HPV E7, HPV L2, HPV 16, HPV E617, HPV L1, HPV16 E6, HPV16 E7, and the like. In some other embodiments, the cell surface antigen is RSV F. In some other embodiments, the antibody targeting an F protein of a RSV virus has sequences of HCDR1, HCDR2 and HCDR3 contained in the heavy chain as shown in SEQ ID NO: 54, and sequences of LCDR1, LCDR2 and LCDR3 contained in the light chain as shown in SEQ ID NO: 56; or has a VH sequence contained in the heavy chain as shown in SEQ ID NO: 54, and a VL sequence contained in the light chain as shown in SEQ ID NO: 56. In some embodiments, the antibody targeting a RSV F protein of a virus-associated antigen has a heavy chain nucleotide sequence as shown in SEQ ID NO: 53 and a light chain nucleotide sequence as shown in SEQ ID NO: 55; or has a heavy chain amino acid sequence as shown in SEQ ID NO: 54 and a light chain amino acid sequence as shown in SEQ ID NO: 56.

In some embodiments, the immunocytokine comprises a heavy chain and a light chain having the following amino acid sequences, respectively: SEQ ID NO: 2 and SEQ ID NO: 8; SEQ ID NO: 6 and SEQ ID NO: 4; SEQ ID NO: 68 and SEQ ID NO: 8; SEQ ID NO: 70 and SEQ ID NO: 4; SEQ ID NO: 10 and SEQ ID NO: 12; SEQ ID NO: 14 and SEQ ID NO: 16; SEQ ID NO: 22 and SEQ ID NO: 24; SEQ ID NO: 26 and SEQ ID NO: 28; SEQ ID NO: 40 and SEQ ID NO: 24; SEQ ID NO: 44 and SEQ ID NO: 28; SEQ ID NO: 46 and SEQ ID NO: 48; SEQ ID NO: 42 and SEQ ID NO: 58; SEQ ID NO: 84 and SEQ ID NO: 86; SEQ ID NO: 60 and SEQ ID NO: 62; SEQ ID NO: 64 and SEQ ID NO: 66; SEQ ID NO: 115 and SEQ ID NO: 116; SEQ ID NO: 117 and SEQ ID NO: 118; SEQ ID NO: 119 and SEQ ID NO: 12; SEQ ID NO: 120 and SEQ ID NO: 16. A person skilled in the art will readily understands that the “heavy chain” of the immunocytokine in the present disclosure refers to a polypeptide chain containing IL-15 or IL-15Ra-fused heavy chain variable region of an antibody targeting a therapy-related cell surface antigen, and the “light chain” of the immunocytokine refers to a polypeptide chain containing IL-15 or IL-15Ra-fused light chain variable region of an antibody targeting a therapy-related cell surface antigen.

In some embodiments, the immunocytokine comprises a heavy chain and a light chain having the following amino acid sequences, respectively: SEQ ID NO:1 and SEQ ID NO: 7; SEQ ID NO: 5 and SEQ ID NO: 3; SEQ ID NO: 67 and SEQ ID NO: 7; SEQ ID NO: 69 and SEQ ID NO: 3; SEQ ID NO: 9 and SEQ ID NO: 11; SEQ ID NO: 13 and SEQ ID NO: 15; SEQ ID NO: 21 and SEQ ID NO: 23; SEQ ID NO: 25 and SEQ ID NO: 27; SEQ ID NO: 39 and SEQ ID NO: 23; SEQ ID NO: 43 and SEQ ID NO: 27; SEQ ID NO: 45 and SEQ ID NO: 47; SEQ ID NO: 41 and SEQ ID NO: 57; SEQ ID NO: 83 and SEQ ID NO: 85; SEQ ID NO: 59 and SEQ ID NO: 61; SEQ ID NO: 63 and SEQ ID NO: 65.

One aspect of the present disclosure provides an immunocytokine, and the immunocytokine includes:

(A) interleukin-15 (IL-15);

(B) an antibody targeting a therapy-related cell surface antigen;

where, IL-15 is fused to N-terminal of the heavy chain constant region and/or N-terminal of the light chain variable region of the antibody directly or via a linker; or IL-15 is fused to C-terminal of the heavy chain constant region and/or a C terminal of the light chain constant region of the antibody.

In some embodiments, the linker is cleavable or non-cleavable. In some embodiments, the linker is independently selected from but not limited GGGGSGGGGSGGGGSG, GSPLGVRGS, GSPLGVR, PLGVR, GGGGSGPLGVRGGGGSG or GGGGSGPLGVR.

In some embodiments, IL-15 is a mammalian IL-15, preferably, a primate IL-15, more preferably, a human IL-15. In some embodiments, the IL-15 is wild-type IL-15 or IL-15 derivatives. In some embodiments, the IL-15 has a nucleotide sequence as shown in SEQ ID NO: 71 or an amino acid sequence as shown in SEQ ID NO: 72; in some embodiments, the IL-15 has a nucleotide sequence as shown in SEQ ID NO: 111 or an amino acid sequence as shown in SEQ ID NO: 112. In some other embodiments, the IL-15 has an amino acid sequence which has a percentage of identity of 85%-100% with SEQ ID NO: 72 or SEQ ID NO: 112.

In some other embodiments, the therapy-related cell surface antigen is a tumor surface antigen. In some other embodiments, the tumor surface antigen is selected from but not limited to EGFR, HER2, HER3, HER4, NY-ESO-1, GPC-3, CLL-1, BCMA, GD2, EpCAM, a chemical chemokine receptor family (CCR1, CCR2, CCR3, CCR4, CCR5, CCR6, CCR7, CCR8, CCR9, CCR10, CCL27, CCL28, CX3CR1, CXCR1, CXCR2, CXCR3, CXCR4, CXCR5, CXCR6), a mucin family (MUC1, MUC2, MUC3A, MUC3B, MUC4, MUC5AC, MUC5B, MUC6, MUC7, MUC8, MUC12, MUC13, MUC15, MUC16, MUC17, MUC19, MUC20), PSMA, CEA, HDAC6, Mesothelin, TERT, TLR, TLR9, TLR4, CD33, GITR, Survivin, CD123, TIGIT, fibroblast growth factor receptors (FGFR), vascular endothelial growth factors (FLT1, KDR/Flk-1, and VEGFR-3), hepatocyte growth factor receptors (HGFR), nerve growth factor receptors (NGFR), insulin-like growth factor receptors (IGFR), platelet-derived growth factor receptors (PDGFR), and hormone receptors (melanocortin 1 receptor (MC1R, MSHR), and the like.

In some other embodiments, the tumor surface antigen is EGFR. In some other embodiments, the antibody targeting EGFR has sequences of HCDR1, HCDR2 and HCDR3 contained in the heavy chain as shown in SEQ ID NO: 32, and sequences of LCDR1, LCDR2 and LCDR3 contained in the light chain as shown in SEQ ID NO: 34; or has a VH sequence contained in the heavy chain as shown in SEQ ID NO: 32, and a VL sequence contained in the light chain as shown in SEQ ID NO: 34. In some embodiments, the antibody targeting the tumor surface antigen EGFR has a heavy chain nucleotide sequence as shown in SEQ ID NO: 29 and a light chain nucleotide sequence as shown in SEQ ID NO: 33; or has a heavy chain amino acid sequence as shown in SEQ ID NO: 30 and a light chain amino acid sequence as shown in SEQ ID NO: 34. In some embodiments, the antibody targeting EGFR has a heavy chain nucleotide sequence as shown in SEQ ID NO: 31 and a light chain nucleotide sequence as shown in SEQ ID NO: 33; or has a heavy chain amino acid sequence as shown in SEQ ID NO: 32 and a light chain amino acid sequence as shown in SEQ ID NO: 34.

In some other embodiments, the tumor surface antigen is HER2. In some other embodiments, the antibody targeting HER2 has sequences of HCDR1, HCDR2 and HCDR3 contained in the heavy chain as shown in SEQ ID NO: 18, and sequences of LCDR1, LCDR2 and LCDR3 contained in the light chain as shown in SEQ ID NO: 20; or the antibody targeting HER2 has a VH sequence contained in the heavy chain as shown in SEQ ID NO: 18, and a VL sequence contained in the light chain as shown in SEQ ID NO: 20. In some embodiments, the antibody targeting HER2 has a heavy chain nucleotide sequence as shown in SEQ ID NO: 17 and a light chain nucleotide sequence as shown in SEQ ID NO: 19; or has a heavy chain amino acid sequence as shown in SEQ ID NO: 18 and a light chain amino acid sequence as shown in SEQ ID NO: 20.

In some embodiments, the immunocytokine comprises a heavy chain and a light chain having the following amino acid sequences, respectively: SEQ ID NO: 90 and SEQ ID NO: 34; SEQ ID NO: 92 and SEQ ID NO: 34; SEQ ID NO: 94 and SEQ ID NO: 34; SEQ ID NO: 96 and SEQ ID NO: 34; SEQ ID NO: 98 and SEQ ID NO: 20; SEQ ID NO: 100 and SEQ ID NO:20; SEQ ID NO: 110 and SEQ ID NO: 102; SEQ ID NO: 110 SEQ ID NO: 104; SEQ ID NO: 98 and SEQ ID NO: 102; SEQ ID NO: 98 and SEQ ID NO: 104; SEQ ID NO: 100 and SEQ ID NO: 102; SEQ ID NO: 100 and SEQ ID NO: 104; SEQ ID NO: 106 and SEQ ID NO: 20; SEQ ID NO: 110 and SEQ ID NO: 108; SEQ ID NO: 106 and SEQ ID NO: 108; SEQ ID NO: 113 and SEQ ID NO: 34; SEQ ID NO: 114 and SEQ ID NO: 34.

In some embodiments, the immunocytokine comprises a heavy chain and a light chain having the following amino acid sequences, respectively: SEQ ID NO: 89 and SEQ ID NO: 33; SEQ ID NO: 91 and SEQ ID NO: 33; SEQ ID NO: 93 and SEQ ID NO: 33; SEQ ID NO: 95 and SEQ ID NO: 33; SEQ ID NO: 97 and SEQ ID NO: 19; SEQ ID NO: 99 and SEQ ID NO: 19; SEQ ID NO: 109 and SEQ ID NO: 101; SEQ ID NO: 109 and SEQ ID NO: 103; SEQ ID NO: 97 and SEQ ID NO: 101; SEQ ID NO: 97 and SEQ ID NO: 103; SEQ ID NO: 99 and SEQ ID NO: 101; SEQ ID NO: 99 and SEQ ID NO: 103; SEQ ID NO: 105 and SEQ ID NO: 19; SEQ ID NO: 109 and SEQ ID NO: 107; SEQ ID NO: 105 and SEQ ID NO: 107.

In one aspect, the present disclosure provides a nucleic acid encoding immunocytokine as described above.

In one aspect, the present disclosure provides a vector containing the nucleic acid sequence of the immunocytokine as described above.

In one aspect, the present disclosure provides a host cell containing the vector as described above.

In one aspect, the present disclosure provides a pharmaceutical composition containing a pharmaceutically acceptable carrier or formulation and the immunocytokine as described above.

In a further aspect, the present disclosure provides a method for treating inflammatory disease or cancer in a subject in need thereof, where the method includes administering to the subject a therapeutically effective amount of a composition containing the immunocytokine in a pharmaceutically acceptable form.

### BRIEF DESCRIPTION OF THE DRAWINGS

Drawings of the description constituting the present application are used to provide a further understanding to the prevent disclosure. The exemplary embodiments and description of the prevent disclosure are intended to explain the prevent disclosure, and don't constitute an improper limitation of the present disclosure. In the drawings:

FIG. 1 is an SDS-PAGE image showing antibody-based immunocytokine constructs (names are referring to Table 1). In each picture of FIG. 1, M denotes a protein marker, A denotes BSIC-01; B denotes BSIC-10; C denotes BSIC-09; D denotes BSIC-05; E denotes BSIC-06; and F denotes BSIC-03. “−” denotes sample loaded without beta-mercaptoethanol; “+” denotes sample loaded with beta-mercaptoethanol.

FIG. 2 (FIG. 2A-FIG. 2F) shows the gel chromatography result of the antibody-based immunocytokine, where FIG. 2A denotes BSIC-03; FIG. 2B denotes BSIC-05; FIG. 2C denotes BSIC-06; FIG. 2D denotes BSIC-09; FIG. 2E denotes BSIC-10; and FIG. 2F denotes BSIC-24.

FIG. 3 shows the ELISA result of the antibody-based immunocytokine binding to IL-15 (FIG. 3A) or IL-15Ra Sushi domain (FIG. 3B), wherein positive 1 denotes IL-15Ra Sushi-Fc fusion protein (Novoprotein); and positive 2 denotes IL-15-Fc fusion protein (GenScript).

FIG. 4 shows the results of Mo7e cell proliferation promoted by antibody-based immunocytokine, where positive 3 denotes a heterodimer formed by IL-15 and IL-15Ra Sushi-Fc via noncovalent interaction.

FIG. 5 shows antibody heavy chain and light chain variable regions of aEGFR (FIG. 5A), aHER2 (FIG. 5B), aPD1 (FIG. 5C), aPDL1 (FIG. 5D and FIG. 5E), and synagis (FIG. 5F), the underlined portion denotes complementary determining regions (CDRs).

FIG. 6 shows the proliferation result of lymphocyte from spleen (6A) and PBMC (6B) stimulated by antibody-based immunocytokine, where positive 3 denotes a heterodimer formed by IL-15 and IL-15Ra Sushi-Fc via noncovalent interaction; negative denotes DPBS.

FIG. 7 shows ELISA result that the antibody-based immunocytokine binds to antigens in vitro; where 7A denotes binding of the immunocytokine to hEGFR; 7B denotes binding of the immunocytokine to hPD-L1; and 7C denotes binding of the immunocytokine to hHER2.

### DETAILED DESCRIPTION OF THE EMBODIMENTS

The present disclosure will be further described herein by citing the following definitions and references to examples. All the contents of the patents and publications mentioned herein, including all the sequences disclosed in these patents and publications, are hereby incorporated by reference.

The “immunocytokines” mentioned herein refer to fusion of cytokines with antibodies.

The term “antibody” herein is used in its broadest sense and includes various kinds of antibody structures, including but not limited to monoclonal antibodies and their derivatives and engineered antibodies, including, for example, multispecific antibodies (such as, DVD-Ig, CrossMab, ART-Ig, FIT-Ig, Duobody and other bispecific antibodies, trispecific antibodies) and antibody fragments, including but not limited to, Fab, Fab′, (Fab)₂, Fv, Diabody, and the like as long as they exhibit the desired binding activity to the therapy-related cell surface antigen, and have a heavy chain variable region and light chain variable region capable of fusion with IL-15 and IL-15Ra at N-terminal. In certain circumstances, for example, when IL-15 or IL-15Ra is fused to C-terminal of a light chain CL or heavy chain CH1, the antibody may also contain the heavy chain constant region or light chain constant region or a portion thereof. In such a circumstance, the antibody may be, for example, in the form of Fab.

The term “CDR” refers to a complementary determining region within the variable region of an immuneglobulin. There are three CDRs in each variable region of the heavy chain and light chain, named CDR1, CDR2 and CDR3, respectively. The term “CDR group” refers to a group of three CDRs that appear in a single variable region capable of binding to an antigen. The exact boundaries of these CDRs have been defined differently according to different systems. The system described by Kabat (Kabat, et al, (1987) and (1991)) not only provides an exact residue numbering system applicable to any variable region of antibodies or binding proteins, but also provides exact residue boundaries defining three CDRs in each heavy chain or light chain sequence. These CDRs can be referred to as Kabat CDR. Chothia and colleagues (Chothia and Lesk (1987) J. Mol. Biol. 196:901-917; Chothia, et at, (1989) Nature 342:877-883) have found that certain sub-portions in Kaba CDRs adopt almost the same peptide framework conformation even though there is great diversity at the amino acid sequence level. These sub-portions are named L1, L2 and L3 or H1, H2 and H3, where “L” and “H” refer to light chain and heavy chain regions, respectively. These regions may be referred to as Chothia CDRs, which have borders that overlap with Kabat CDRs. Other boundaries defining CDRs that overlap with Kabat CDRs have been described by Padlan (1995) FASEB J. 9:133-139 and MacCallum (1996) J. Mol. Biol. 262(5):732-45). There are other CDR boundary definitions that may not strictly follow one of the systems herein, but will still overlap with Kabat CDR, although they can be shortened or lengthened in view of the predictions or experiments that a specific residue or residue group or even the whole CDR does not significantly influence antigen binding. The methods of CDR definition by Kabat and Contact are combined herein (see details on http://www.bioinf.ore.uk/abs/).

The term “homology” or “identity” refers to the identity of primary sequence between two polymer molecules (for example, between two nucleic acid molecules (e.g., between two DNA molecules or two RNA molecules) or two polypeptide molecules). When a certain position of the primary sequence in these two molecules is occupied by a same monomeric c subunit, for example, if a certain position in each molecule of two DNA molecules is occupied by adenine, the two molecules are homologous or same at that position. Identity between two sequences varies directly with the number of matching positions or homologous positions. For example, if half of the positions in two sequences (for example, 5 positions in a polymer of 10 subunits in length) are homologous, the two sequences are 50% homologous (also called 50% homology or 50% identity); if 90% of the positions (for example, 9 positions in 10 positions) are matched or homologous, the two sequences are 90% homologous (also called 90% homology or 90% identity).

The “IL-15” of the present disclosure is a mammalian interleukin 15 (IL-15), preferably, a primate IL-15, more preferably a human IL-15. The “IL-15” of the present disclosure refers to an IL-15 derivative or variant which has an amino acid sequence having at least a percentage of identity of 85% (namely, equivalent to about 20 amino acid changes), preferably at least a percentage of identity of 99% (change of about 1 amino acid) to the amino acid sequence as shown in SEQ ID NO: 72 while still maintaining equivalent physiological activity to that of a wild-type IL-15. A person skilled in the art can identify such a derivative based on the knowledge and the technical teaching of the disclosure. It should also be understood that natural amino acids may be substituted by chemically modified amino acids. Usually, such chemically modified amino acids have an improved polypeptide half-life (see WO/2015/131994). With regard to the definition of the “percentage of identity” between the two amino acid sequences used previously, see WO/2015/131994.

Preferably, the IL-15 derivative or variant may be an IL-15 agonist or superagonist. A person skilled in the art can identify the IL-15 agonist or superagonist based on the method of the prior art. As examples of the IL-15 agonists or superagonists, those disclosed in WO2005/085282 or Zhu, et al. (J. Immunol, vol. 183 (6), p:3598-607, 2009) can be cited.

More preferably, the IL-15 agonist or superagonist includes a substitution selected from the following group consisting L45D, L45E, 551 D, L52D, N72D, N72E, N72A, N72S, N72Y and N72P (the amino acid positions above are referring to human IL-15 as shown in SEQ ID NO.: 72).

The “IL-15Ra Sushi domain” of the present disclosure has the general meaning well known in the art. The Sushi domain is the Sushi domain of mammalian IL-15Ra, preferably, Sushi domain of a primate IL-15Ra, and more preferably, Sushi domain of a human IL-15Ra. Preferably, the sushi domain containing human IL-15Ra has an amino acid sequence as shown in SEQ ID NO.: 74.

As used herein, the term “derivative of the IL-15 sushi domain” in the present disclosure refers to an amino acid sequence with at least a percentage of identity of 85% (namely, equivalent to about 10 amino acid changes), preferably a derivative with at least a percentage of identity of 99% (about 1 amino acid change) to the amino acid sequence as shown in SEQ ID NO: 74. A person skilled in the art can identify such a derivative based on his personal knowledge and the technical teaching of the disclosure. It should also be understood that natural amino acids may be substituted by chemically modified amino acids. Usually, such chemically modified amino acids have improved polypeptide half-life (see WO/2015/131994). With regard to the definition of the “percentage of identity” between the two amino acid sequences used previously, see, for example, WO/2015/131994.

The “therapy-related cell surface antigen” of the present disclosure refers to an antigen expressed on the surface of a cell that can be targeted for IL-15-mediated treatment of diseases. The “therapy-related cell surface antigen” of the present disclosure includes CD antigens, immune checkpoint antigens, tumor-associated antigens, tumor specific antigens, and virus-induced tumor antigens, and may be selected from but not limited to the following antigens: CD19, PD-1, PD-L1, HER2, STATS, STEAP1, CTLA-4, IDO, NY-ESO-1, CD40, CSF1R, BCMA, MUC1, ADORA2A, CD20, GD2, TLR7, WT1, IFNAR1, CD47; Neoantigen, EGFR, LAG-3, OX40, PSMA, Mesothelin, TERT, TLR, TLR9, 4-1 BB, IL2R, TLR4, CD33, GITR, HPV E6, Survivin, CD123, TIGITTIM-3, CD73, HPV E7, TLR3, CD38, EBV, STING, CD22, GPC3, HDAC1, CXCR4, GMCSFR, CD30, CEACAM5, HDAC6, HPV, CD3, MAGE-A3, TNF, PSA, CD25, CEA, EPCAM, CMV, IL12, PRAME, IL12R, 5T4, Beta catenin, CCR2, PMEL, CXCL12, IGF1, CD46, CXCR1, GMCSF, IL15R, ROR1, TGFBR2, CCR4, FLT-3, FOLR1, GCSFR, ICOS, JAK2, KRAS, VISTA, CD133, CD27, CD39, CEACAM6, NKG2D, STATS, TGFB1, TLR2, USP7, ANG1, ANG2, B7-H3, CLEC12A, IL13RA2, RIG-I, TRP2, VEGF, AFP, Alpha-Gal, COX-2, EPHA2, gp96, MUC16, p53, TGF-β, CD138, CDw136, CS1, CXCR2, EGFRvIII, Gelactin-3, Globo H, GR, IFNAR2, IFNGR1, IL6, JAK1, MLANA, RAS, SLAMF7, TDO, TGFB2, TLR8, ALK, Arginase, CCR1, CD56, CD70, FAP, GD3, IDH1, IL6R, IRAK4, MAGE-A4, MERTK, MIF, PSCA, PTGER4, SIRPA, TGFB, TGFBR1, ACPP, ADORA2B, AR, Brachyury, CA19-9, CD32, CEACAM1, Gastrin, HDAC, HPV L2, IFNAR, IFNGR, IGF1R, IGF2, IL15, IL17R, IL1 B, IL7R, JAK, MAGE-A, MAGE-A1, MAGE-A6, P38, RORC, TLR5, VEGFR2, ADORA3, ATRT, B7-H4, c-KIT, CCR7, CD11b, CD135, CD171, CD174, CDH3, CX3CR1, Gelactin-1, GM3, HLA-A2, HSP70, MO, IL17, IL2RB, JAK3, MDAS, NKG2A, PBF, PVRIG, SPAM1, URLC10, VEGFR1, ABCB5, ADABP, ADAM17, ADP, AEG1, Alpha-lactalbumin, AMHR2, ASPH, AXL, BCL2, BTE6-LX-8b, BTE6-X-15-7, Carbohydrate antigens, CCL20, CCL3, CCNB1, CD147, CD155, CD16, CD162, CD16a, CD200, CD21, CD28, CD44, CD52, CD54, CD7, CD80, CD88, Claudin 18, cMET, COX2, CSF1, CTCFL, CXCR5, CXCR7, E1A, EIF2AK3, ERG, FGF2, FN1, GC, GM2, gpA33, HBV, Hemagglutinin, HER3, HILPDA, HLA-DR, HMW-MAA, HP59, HPV 16, HPV E6/7, HPV L1, HSP105, HSP65, HVEM, Hyaluronan, IL13RA1, IL2, IL21R, IL8, KIF20A, KIR2DL1, KIR2DL3, LXR, MAGE-A10, MAGE-C2, Mammaglobin A, MAPK, MICA, MiHA, MMP-11, MVP, Myeloblastin, N-Myc, NKp46, NLRP3, NR2F6, Oncofetal Antigen, P2RX7, RhoC, SIM-2, SSTR2, SSX2, STAT1, STn, TAG72, TAMA, TFDP3, TGFBR, TSA, TYK2, Tyrosinase, VEGFA, 5′ Nucleotidase (Ecto 5′ Nucleotidase or CD73 or NT5E or EC 3.1.3.5), ADAM9, AIM2, B7-H6, BAFF-R, BAI1, BARD1, BOB-1, CA9, Cancer testis antigen, CB2, CBLB, CCR9, CD13, CD130, CD150, CD160, CD200R1, CD267, CD29, CD3E, CD4, CD51, CD8, CGEN-XXXX, Claudin 6, CLEC2D, COX, COX-1, CPEB4, CPEG4, CRBN, CRLF2, CSPG4, CTA, CXCL1, CXCR3, Cytosine deaminase, DCK, DKK1, DLL3, DR3, DR5, EBNA3C, EGF, EGFRS, ELVAL4, EPHA3, EPS8, EVI1, FAIM-3, FasR, FCU1, FLT3, FOLR, FOXM1, FSHR, Galectin-3, GalNAc, GARP, Gelactin-9, Gelatcin-1/3/9, GLD18, GNRHR, GP160, GP73, H3.3K27M, HAGE, HDAC2, HDAC8, HPV16 E6, HPV16 E7, HSP, Hypoxia, ICAM, ICAM7, ID01, IFNG, IFNGR2, IGF2R, IGFBP2, IGK, IL10 RA, IL12RB1, IL13, IL13R, IL13Ralpha2, IL15 RA, IL17A, IL17B, IL1A, IL1R1, IL1R3, IL21, IL22R, IL27R, IL2RA, IL35, IL9R, Integrin beta-7, IRAK1, ITGB5, Kappa myeloma antigen, KIR2DL2, Kynurenine, L1CAM, Lambda Myeloma Antigen, LAMP, LLO, LXRA, LXRB, Mas receptor, MG7, MHCI, MHCII, MIC, MOSPD2, MRP-3, MRP1, MRP3765, muGNTP01, MYB, MYBL2, NFAT, NGcGM3, Nrf2, p38 MAP Kinase (EC 2.7.11.24), P55, PAM4, PAP, PASD1, PCDH18, PD-L2, PI3K-delta, POTE, PPT, Protein tolemerase, PTGER2, RANKL, RBL001, RNF43, ROR2, S100A9, SEREX, SLAMF1, STAT, TACSTD2, TASTD2, TDO2, TEM, thymidine kinase, Thymidylate synthase, TIE2, TIMP3, TM4SF5, TOP1, TRBC1, TRBC2, TRIF, Tryptophan, TSHR, TWEAK, UTA2-1, VDBP, VRP, VSIG-4, XAGE1, XAGE1A, ZP1, and ZP3.

The term “linker” of the present disclosure refers to two amino acid residues or a polypeptide with two or more amino acid residues used to fusing two polypeptides. Such a linker is well known in the art (see, for example, Holliger, et al. (1993) Proc. Natl. Acad. Sci. USA 90:6444-6448; Poljak, et al. (1994) Structure 2:1121-1123). Suitably, the non-immunogenic linker includes, for example, GS, (G4S)n, (SG4)n, (G4S)n or G4 (SG4)n peptide linker. “n” is generally 1-10, and usually 2-4. In some embodiments, the linker may be cleavable linker, for example, a self-cleavable, enzymatically cleavable or chemically cleavable. The entire linker in WO2011/034605 is incorporated herein by reference. Enzymatical cleavage may be performed by, but no limited to endopeptidases or exopeptidases. Non-limiting examples of endopeptidases include MMP2 (matrix metalloproteinase 2), urokinase, Lys-C, Asp-N, Arg-C, V8, Glu-C, chymotrypsin, trypsin, pepsase, papain, thrombin, Genenase, factor Xa, TEV (tobacco etch virus cysteine protease), enterokinase, HRV C3 (human rhinovirus C3 protease), ininogenase, Bacillus subtilis protease-like proprotein convertase (for example, Furin (PC1), PC2 or PC3) and N-arginine dibasic convertase. Non-limiting examples of exopeptidases include carboxypeptidase A, carboxypeptidase B, carboxypeptidase D, carboxypeptidase E (also called carboxypeptidase H), carboxypeptidase M, carboxypeptidase N or carboxypeptidase Z.

In some embodiments, chemical cleavage may be performed by hydroxylamine, N-chlorosuccinimide, N-bromosuccinimide or cyanogen bromide.

### EXAMPLES

It should be indicated that in the case of no conflict, examples in the present application are only illustrative, and are not intended to limit the present disclosure in any way.

### EXAMPLES

### Example 1 Construction of an Antibody-Based Immunocytokine

Gene segments encoding heavy chain (H) and light chain (L) of anti-EGFR antibody (aEGFR), Palivizumab (Syn), anti-PD-1 antibody (a PD-1), anti-PD-L1 antibody (a PD-L1), anti-HER2 antibody (aHER2), IL-15, IL-15Ra, or IL-15RaSushi were synthesized, respectively. IL-15 was fused to N-terminal of heavy chain variable region of the antibody or C-terminal of CH1, while IL-15Ra was fused to N-terminal of light chain variable region or C-terminal of light chain, or IL-15 was fused to N-terminal of light chain variable region or a C-terminal of light chain, while IL-15Ra was fused to N-terminal of heavy chain variable region or C-terminal of CH1 by standard molecular biology techniques. All the sequences were verified by sequencing (see sequences in Table 1).

TABLE 1
Sequences of the antibody-based immunocytokine

| | | | Nucleotide | Amino acid |
|---|---|---|---|---|
| | | | sequence | sequence |
| Construct | Protein | Composition | Seq ID No: | Seq ID No: |
| BSIC-1 | IL-15-SynH(null): | Chain-1 | 1 | 2 |
| | IL-15RaSushi-SynL | Chain-2 | 7 | 8 |
| BSIC-2 | IL-15RaShi-SynH(null): | Chain-1 | 5 | 6 |
| | IL-15-SynL | Chain-2 | 3 | 4 |
| BSIC-3 | IL-15-aHER2H(null): | Chain-1 | 9 | 10 |
| | IL-15RaSushi-aHER2L | Chain-2 | 11 | 12 |
| BSIC-4 | IL-15RaSushi-aHER2H(null): | Chain-1 | 13 | 14 |
| | IL-15-aHER2L | Chain-2 | 15 | 16 |
| BSIC-5 | IL-15-aEGFRH: | Chain-1 | 21 | 22 |
| | IL-15RaSushi-aEGFRL | Chain-2 | 23 | 24 |
| BSIC-6 | IL-15RaSushi-aEGFRH: | Chain-1 | 25 | 26 |
| | IL-15-aEGFRL | Chain-2 | 27 | 28 |
| BSIC-7 | IL-15-aEGFRH(null): | Chain-1 | 39 | 40 |
| | IL-15RaSushi-aEGFRL | Chain-2 | 41 | 42 |
| BSIC-8 | IL-15RaSushi-aEGFRH(null): | Chain-1 | 43 | 44 |
| | IL15-aEGFRL | Chain-2 | 27 | 28 |
| BSIC-9 | IL-15-aPDL1H(null): | Chain-1 | 45 | 46 |
| | IL-15RaSushi-aPDL1L | Chain-2 | 47 | 48 |
| BSIC-10 | IL-15RaSushi-aPDL1H(null): | Chain-1 | 49 | 50 |
| | IL15-aPDL1L | Chain-2 | 51 | 52 |
| BSIC-11 | IL-15RaSushi-aPDL1H(null): | Chain-1 | 87 | 88 |
| | IL-15-aPDL1L | Chain-2 | 57 | 58 |
| BSIC-12 | IL-15-aPD1H(null): | Chain-1 | 83 | 84 |
| | IL-15RaSushi-aPD1L | Chain-2 | 85 | 86 |
| BSIC-13 | Syn(null) | Chain-1 | 53 | 54 |
| | | Chain-2 | 55 | 56 |
| BSIC-14 | IL-15-aPDL1H: | Chain-1 | 59 | 60 |
| | IL-15RaSushi-aPDL1L | Chain-2 | 61 | 62 |
| BSIC-15 | IL-15RaSushi-aPDL1H(null): | Chain-1 | 63 | 64 |
| | IL-15-aPDL1L | Chain-2 | 65 | 66 |
| BSIC-16 | IL-15-SynH: | Chain-1 | 67 | 68 |
| | IL-15RaSushi-SynL | Chain-2 | 7 | 8 |
| BSIC-17 | IL-15RaSushi-SynH: | Chain-1 | 69 | 70 |
| | IL-15-SynL | Chain-2 | 3 | 4 |
| BSIC-18 | aPDL1-1 | Chain-1 | 79 | 80 |
| | | Chain-2 | 81 | 82 |
| BSIC-19 | aHER2 | Chain-1 | 17 | 18 |
| | | Chain-2 | 19 | 20 |
| BSIC-20 | aEGFR | Chain-1 | 29 | 30 |
| | | Chain-2 | 33 | 34 |
| BSIC-21 | aPD1 | Chain-1 | 75 | 76 |
| | | Chain-2 | 77 | 78 |
| BSIC-22 | aEGFR(null) | Chain-1 | 31 | 32 |
| | | Chain-2 | 33 | 34 |
| BSIC-23 | aPDL1(null) | Chain-1 | 35 | 36 |
| | | Chain-2 | 37 | 38 |
| BSIC-24 | IL-15-16GS-aHER2H | Chain-1 | 19 | 20 |
| | | Chain-2 | 9 | 10 |
| BSIC-25 | IL-15-16GS-aHER2L | Chain-1 | 17 | 18 |
| | | Chain-2 | 15 | 16 |
| BSIC-26 | IL-15RaSushi-SynL | Chain-1 | 53 | 54 |
| | | Chain-2 | 7 | 8 |
| BSIC-27 | IL-15RaSushi-SynH(null) | Chain-1 | 5 | 6 |
| | | Chain-2 | 55 | 56 |
| BSIC-28 | IL-15-16GS-SynH(null) | Chain-1 | 1 | 2 |
| | | Chain-2 | 55 | 56 |
| BSIC-29 | IL-15-MP2GS-aEGFRH(null) | Chain-1 | 89 | 90 |
| | | Chain-2 | 33 | 34 |
| BSIC-30 | IL-15-MP2-aEGFRH(null) | Chain-1 | 91 | 92 |
| | | Chain-2 | 33 | 34 |
| BSIC-31 | aEGFRH(null)-MP2GS-IL-15 | Chain-1 | 93 | 94 |
| | | Chain-2 | 33 | 34 |
| BSIC-32 | aEGFRH(null)MP2-IL-15 | Chain-1 | 95 | 96 |
| | | Chain-2 | 33 | 34 |
| BSIC-33 | IL-15WT-MP2GS-aHER2H(null) | Chain-1 | 97 | 98 |
| | | Chain-2 | 19 | 20 |
| BSIC-34 | IL-15WT-MP2-aHER2H(null) | Chain-1 | 99 | 100 |
| | | Chain-2 | 19 | 20 |
| BSIC-35 | IL-15WT-MP2GS-aHER2L | Chain-1 | 109 | 110 |
| | | Chain-2 | 101 | 102 |
| BSIC-36 | IL-15WT-MP2-aHER2L | Chain-1 | 109 | 110 |
| | | Chain-2 | 103 | 104 |
| BSIC-37 | IL-15WT-MP2GS-aHER2H(null): | Chain-1 | 97 | 98 |
| | IL-15WT-MP2GS-aHER2L | Chain-2 | 101 | 102 |
| BSIC-38 | IL-15WT-MP2GS-aHER2H(null): | Chain-1 | 97 | 98 |
| | IL-15WT-MP2-aHER2L | Chain-2 | 103 | 104 |
| BSIC-39 | IL-15WT-MP2-aHER2H(null): | Chain-1 | 99 | 100 |
| | IL-15WT-MP2GS-aHER2L | Chain-2 | 101 | 102 |
| BSIC-40 | IL-15WT-MP2-aHER2H(null): | Chain-1 | 99 | 100 |
| | IL-15WT-MP2-aHER2L | Chain-2 | 103 | 104 |
| BSIC-41 | IL-15WT-16GS-aHER2H(null) | Chain-1 | 105 | 106 |
| | | Chain-2 | 19 | 20 |
| BSIC-42 | IL-15WT-16GS-aHER2L | Chain-1 | 109 | 110 |
| | | Chain-2 | 107 | 108 |
| BSIC-43 | IL-15WT-16GS-aHER2H(null): | Chain-1 | 105 | 106 |
| | IL-15WT-16GS-aHER2L | Chain-2 | 107 | 108 |
| BSIC-44 | hIL15WT-16GS-aEGFRH | Chain-1 | / | 113 |
| | | Chain-2 | / | 34 |
| BSIC-45 | aEGFRH-16GS-IL15WT | Chain-1 | / | 114 |
| | | Chain-2 | / | 34 |
| BSIC-46 | aEGFR-FabL-hIL15RSUSHI: | Chain-1 | / | 115 |
| | aEGFR-FabH-hIL15 | Chain-2 | / | 116 |
| BSIC-47 | aEGFR-FabL-hIL15RSUSHI: | Chain-1 | / | 117 |
| | aEGFR-FabH-hIL15 | Chain-2 | / | 118 |
| BSIC-48 | IL15-aHER2H: | Chain-1 | / | 119 |
| | IL15RaSushi-aHER2L | Chain-2 | 11 | 12 |
| BSIC-49 | IL15RaSushi-aHER2H: | Chain-1 | / | 120 |
| | IL15-aHER2L | Chain-2 | 15 | 16 |
| IL-15 | IL-15 | / | 71 | 72 |
| IL-15RaSushi | IL-15RaSushi | / | 73 | 74 |
| IL-15Ra | IL-15Ra | / | 87 | 88 |
| IL-15WT | | | 111 | 112 |

Note:
null denotes that Fc had no ADCC and CDC function or had weakened ADCC and CDC functions.

### Example 2 Expression, Purification and Size-Exclusion Chromatography (SEC) of the Antibody-Based Immunocytokine

The heavy chain and light chain expression vectors for immunocytokine constructed in Example 1 were transiently co-transfected into Expi 293 cells (ThermoFisher) with a molar ratio of 1:1:40 ml Expi293 (3-4×10⁶ cell/ml) were seeded in a 125 ml cell culture flask, 80 ug plasmid were diluted with 2 ml Opti-MEM (Invitrogen), and then added to 2 ml Opti-MEM containing 120 μl PEI(Polysciences). After standing at room temperature for 30 min, the plasmid-PEI mixture was added to the cell culture for culture at 125 rpm, 37° C., 5% CO₂. Cell culture supernatant was collected at 96 h after transfection, purified by Protein A Resin (Genscript), and then analyzed by SDS-PAGE.

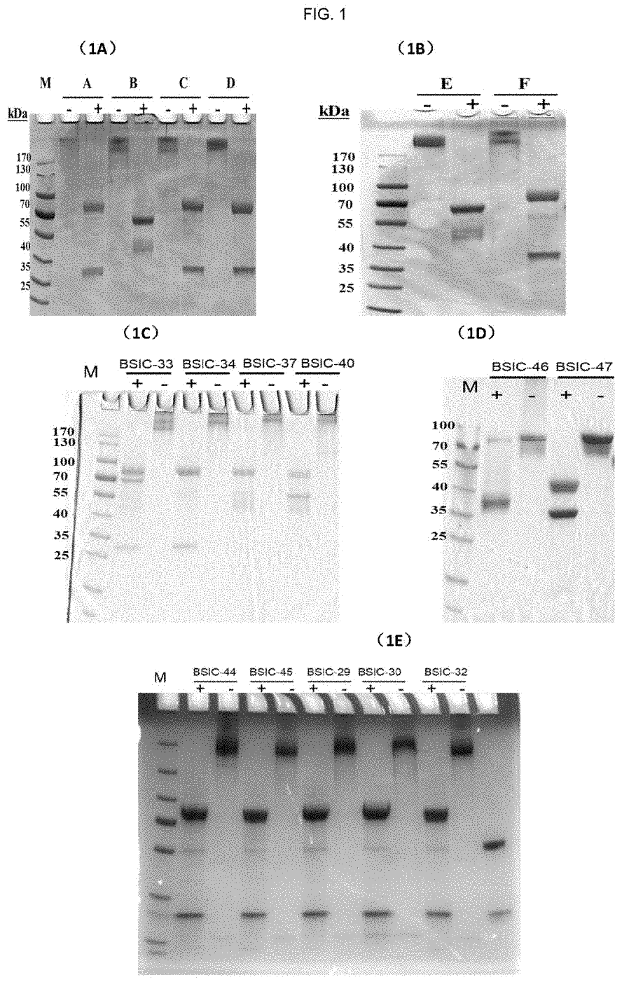

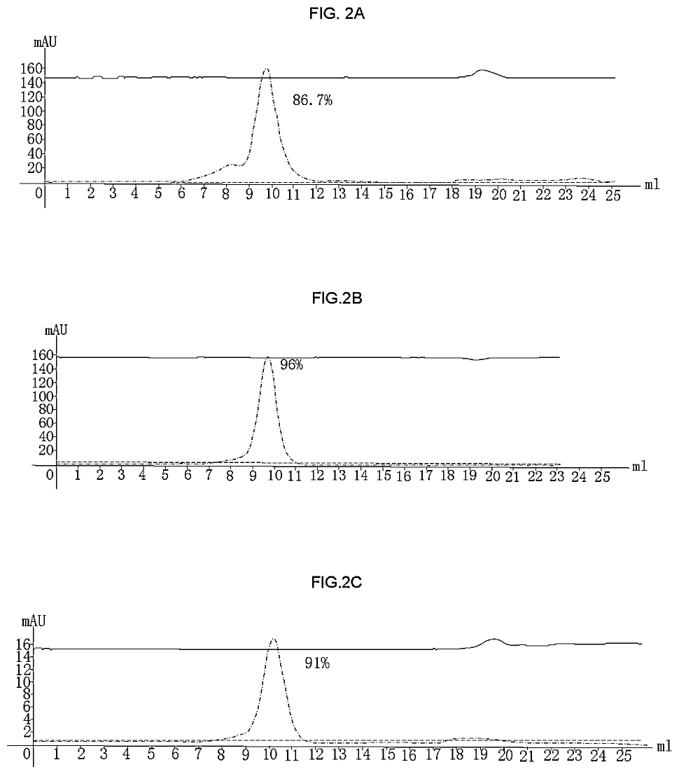

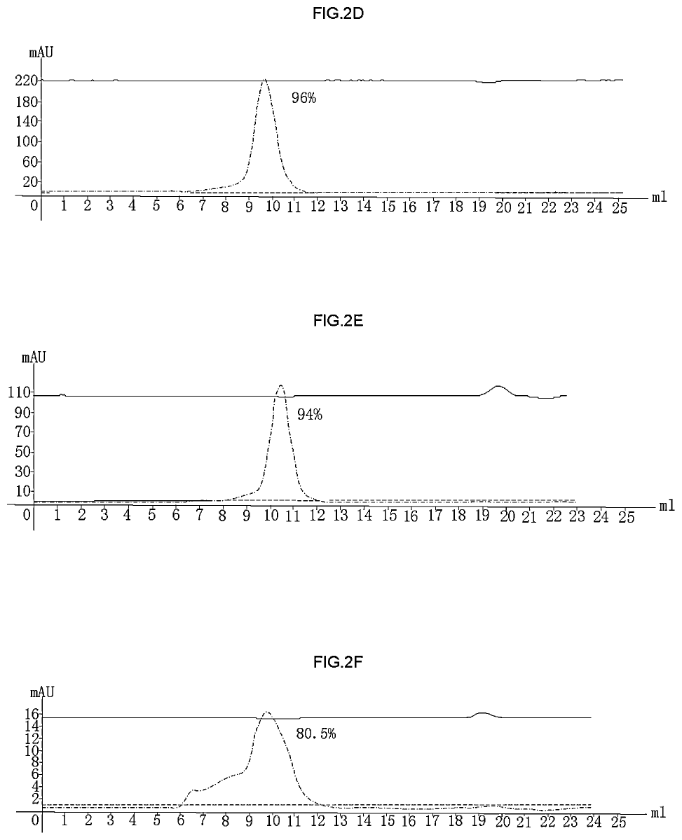

The purified immunocytokine by Protein A resin was subjected to chromatographic analysis via GE's AKTA, and the chromatographic column used was: Superdex 200 Increase 10/300 GL size exclusion chromatographic column. The solution for the size exclusion chromatography was PBS buffer (0.010 M phosphate buffer, 0.0027 M KCl, 0.14 M NaCl, pH=7.4). SDS in FIG. 1 and chromatogram in FIG. 2 indicated that the immunocytokine complex had considerable purity and can be effectively separated by a chromatographic column.

### Example 3 In Vitro Activity Evaluation of the Antibody-Based Immunocytokine

3.1 IL-15 or IL-15RaSushi Binding Activity

hIL-15 (SinoBiological) or IL-15RaSushi (Miltenyi) (100 ng/well) (DPBS buffer, pH=7.4) was coated in a 96-well plate and incubated overnight at 4° C. After blocked by DPBST containing 2% skimmed milk powder for 1 h at room temperature, and washed for 3 times with DPBS containing 0.05% Tween-20, the gradient diluted immunocytokines were added and incubation for 2 h at room temperature. After washed for 4-5 times with DPBS containing 0.05% Tween-20, HRP conjugated anti-human kappa light chain (Cat. A18853, Thermo Fisher Scientific, 1:2000) secondary antibody was added for incubation for 2 h at room temperature, then washed for 4-5 times with DPBS containing 0.05% Tween-20. TMB reagent (BioLegend) was used for color development, and readings was performed at OD450. Data was analyzed by nonlinear regression with a log(agonist) vs. response model via Prizm Graphpad software.

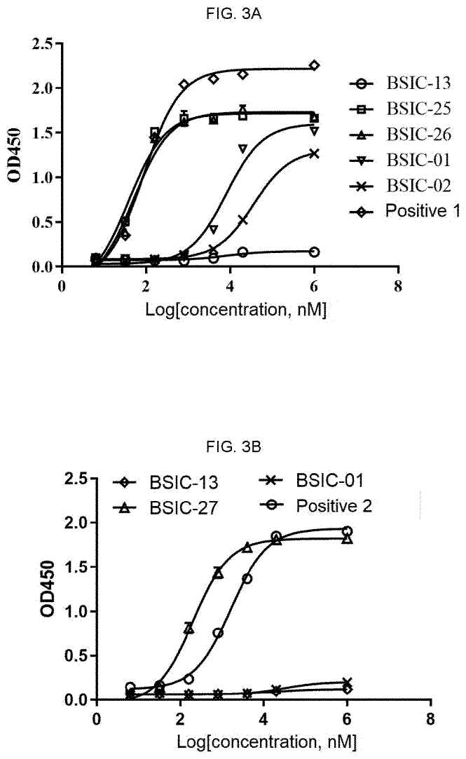

As shown in FIG. 3, BSIC-01 (IL15-SynH(null): IL15RaSushi-SynL) and BSIC-02 (IL15RaShi-SynH(null): IL15-SynL) had relatively low (3A) affinity to IL-15 when compared to positive-1(IL-15RaSushi-Fc), indicating that the IL-15 and IL-15RaSushi in BSIC-01 (IL 15-SynH(null):IL15 RaSushi-SynL) or BSIC-02 (IL 15RaShi-SynH(null): IL 15-SynL) formed a stable complex. BSIC-01 (IL15-SynH(null): IL 15RaSushi-SynL) had relatively low (3B) affinity to IL-15 consistent with the framework antibody BSIC-13Syn(null) when compared to positive-2(IL-15-Fc), indicating that IL-15 and IL-15RaSushi in BSIC-01 (IL 15-SynH(null): IL 15RaSushi-SynL) formed a very stable intramolecular complex. BSIC-27 (IL-15RaSushi-SynH(null)) had a higher affinity to IL-15RaSushi.

3.2 Antigen Binding Activity

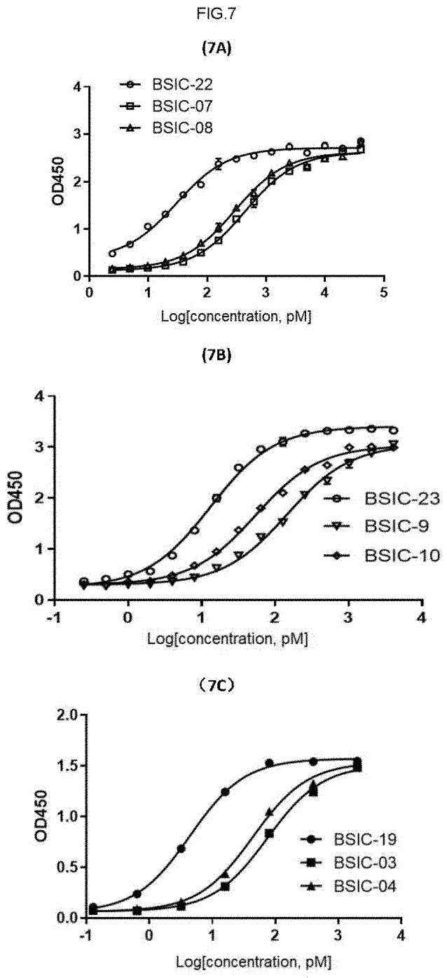

hEGFR-his (SinoBiological), hHER2(ACRO) or hPDLI (100 ng/well) were coated in a 96-well plate and incubated overnight at 4° C. After blocked by PBST (0.5% Tween-20 in PBS) containing 2% skim milk powder for 1 h at room temperature, the gradient diluted immunocytokines were added and incubated for 2 h at room temperature. After washed for 4-5 times with PBST containing 2% skim milk powder, HRP anti-human kappa light chain (Invitorgen) secondary antibody was added and incubated for 1 h at room temperature, then washed for 4-5 times with PBST containing 2% skim milk powder. Color development was performed with TMB reagent (BioLegend, Cat. 421101), and readings were performed at 450 nm. Or color development was performed with QuantaBlu fluorescent peroxidase substrate (Life technologies, Cat. 15169), and reading was performed at 325 nm and 420 nm. Data was analyzed by nonlinear regression with a specific binding model via Prizm Graphpad software. As shown in FIG. 7; the antibody-based immunocytokine had better affinity to antigens.

### Example 4 Mo7e Proliferation-Promoting Assay

Mo7e cells (Cell Resource Center, IBMS, CAMS/PUMC) were cultured in RPMI 1640 medium with 10% fetal bovine serum, 10 ng/ml GM-CSF(Peprotech), and two antibiotics (penicillin-streptomycin). Cell viability should exceed 95% as detected by trypan blue before assay. Cells were washed for twice with RPMI 1640(10% FBS+ two antibiotics (penicillin-streptomycin)), pended to density of 40,000/100 μl, and then added (50 μl/well) to a 96-well plate (opaque black bottom). RPMI 1640(10% FBS+ two antibiotics (penicillin-streptomycin)) medium containing the immunocytokines was prepared according to the pre-set concentration. The prepared medium containing the immunocytokine was added (50 μl/well) to the 96-well plate (three replicates for each gradient). Afterwards, the 96-well plate was gently shaken to make the cytokine cells distributed evenly, and then incubated. 72 h later, color development was performed by MTS (Promega), and reading was performed at OD490. Or color development was performed by Cell TiterGLO (Promega), and luminescent was read. Data was analyzed by nonlinear regression with a log(agonist) vs. response model via Prizm Graphpad software.

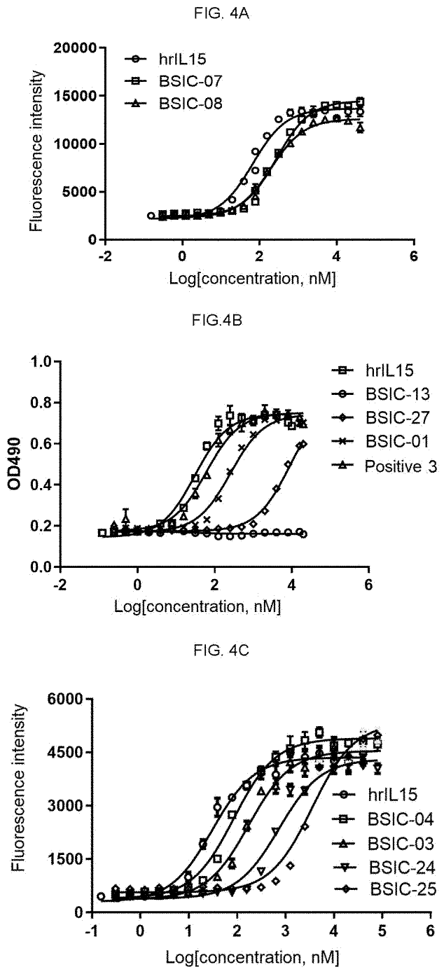

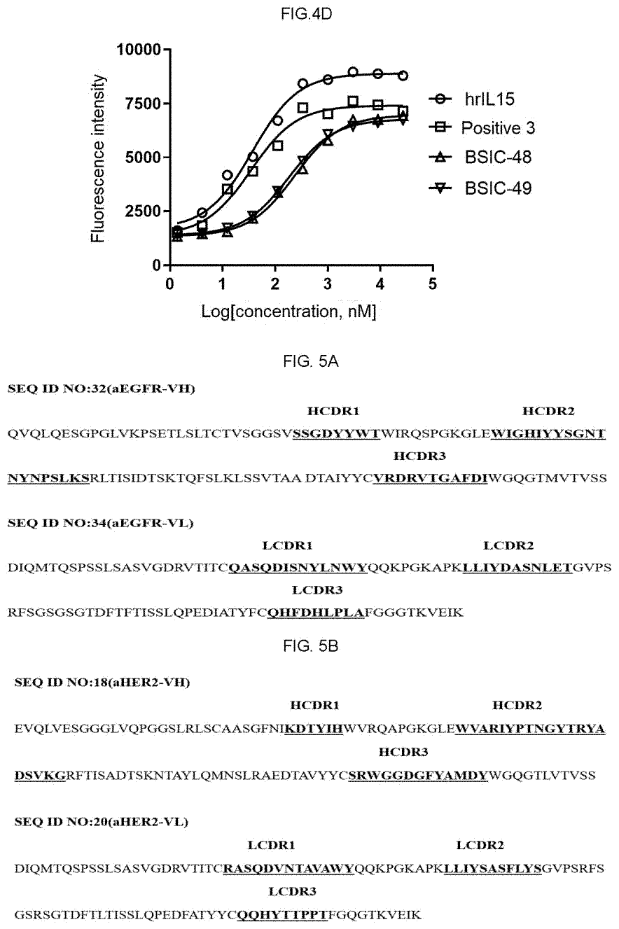

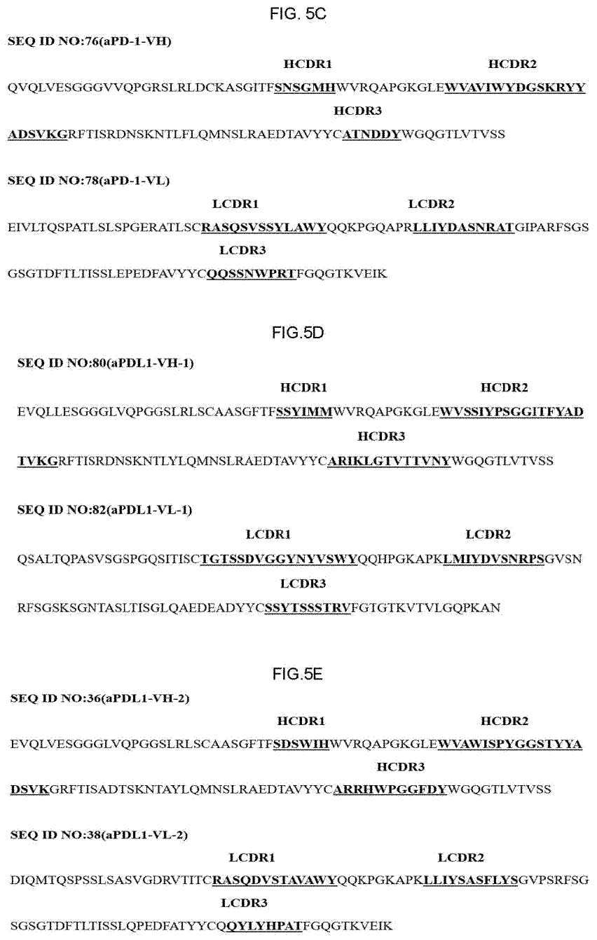

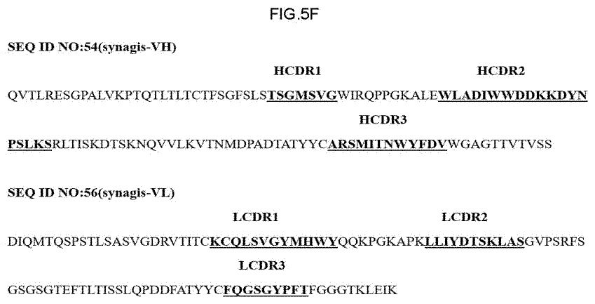

As shown in FIG. 4. The proliferation-promoting effect of BSIC-01 (IL 15-SynH(null): IL15 RaSushi-SynL) on Mo7e proliferation was weaker than that of positive-3 (in which IL-15 and IL-15RaSushi-Fc formed a heterodimer via noncovalent interaction) or rhIL-15, but stronger than BSIC-27 (FIG. 4A), indicating that the intramolecular complex formed by IL-15 and IL-15Rasushi via noncovalent interaction may effectively promote the proliferation of Mo7e cells. BSIC-07 (IL15-aEGFRH(null): IL 15RaSushi-aEGFRL) and BSIC-08(IL15RaSushi-aEGFRH(null): IL 15-aEGFRL) had similar proliferation-promoting effect on Mo7e, although slightly weaker than that of rhIL-15. The promoting effect of BSIC-03 (IL15-aHER2H(WT): IL15RaSushi-aHER2L) and BSIC-04 (IL15RaSushi-aHER2(WT): IL15-aHER2L) on Mo7e proliferation was stronger than that of BSIC-24 (IL-15-a HER2H(WT)) or IL-15-aHER2L(WT) (IL-15-aHER2H(WT)), consistent with results from Palivizumab or aEGFR-based constructs.

### Example 5 Lymphocyte Activation In Vivo

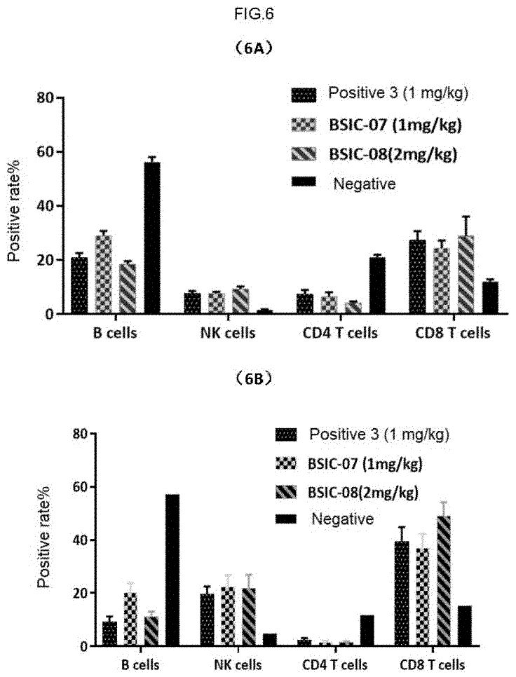

DPBS or immunocytokine was intraperitoneally injected into female C57BL/6 mice (6-7 weeks). Four days later, venous blood was collected from the orbital socket to isolate PBMCs, at the same time, mice were sacrificed and the spleens were taken for preparation of -splenocytes suspension The population percentage of different immune cells in PBMC and spleen was analyzed by flow cytometry with antibodies as follows: PE rat anti-mouse CD19, APC rat anti-mouse CD45, FITC rat anti-mouse CD335, FITC rat anti-mouse CD3, PE rat anti-mouse CD4, PE rat anti-mouse CD8, TruStain FcX plus (anti-mouse CD 16/32)(Biolegend). As shown in FIG. 6, the antibody-based immunocytokine could effectively activate CD8+ T cells and NK cells in mice.

What is claimed is:

1. An immunocytokine, comprising:
(A) interleukin-15 (IL-15);
(B) interleukin-15 receptor-a subunit (IL-15Ra);
(C) an antibody targeting a therapy-related cell surface antigen;
wherein:
IL-15 is fused to N-terminal of heavy chain variable region of the antibody with or without a linker while IL-15Ra is fused to N-terminal of light chain variable region of the antibody with or without a linker; or,
IL-15 is fused to N-terminal of light chain variable region of the antibody with or without a linker while IL-15Ra is fused to N-terminal of heavy chain variable region of the antibody with or without a linker; or
IL-15 is fused to C-terminal of light chain constant region (CL) of the antibody with or without a linker while IL-15Ra is fused to C-terminal of heavy chain constant region (CH1) of the antibody with or without a linker; or
IL-15 is fused to C-terminal of heavy chain constant region (CH1) of the antibody with or without a linker while IL-15Ra is fused to a C-terminal of light chain constant region (CL) of the antibody with or without a linker.

2. The immunocytokine of claim 1, wherein IL-15 is a wild-type IL-15 or an IL-15 derivative.

3. The immunocytokine of claim 2, wherein the IL-15 has an amino acid sequence which has a percentage of identity of 85%-100% with SEQ ID NO: 72 or SEQ ID NO: 112, or consists of such an amino acid sequence.

4. The immunocytokine of claim 1, wherein IL-15Ra is a full-length IL-15Ra, sushi domain of IL-15Ra or a derivative of IL-15Ra Sushi domain.

5. The immunocytokine of claim 1, wherein IL-15Ra has an amino acid sequence which has a percentage of identity of 85%-100% with SEQ ID NO: 74 or SEQ ID NO: 88, or consists of such an amino acid sequence.

6. The immunocytokine of claim 1, wherein the therapy-related cell surface antigen is an immune checkpoint protein or a tumor antigen;
preferably, the therapy-related cell surface antigen is selected from: an epidermal growth factor receptor family (EGFR, HER2, HER3, HER4), PD-1, PD-L1, STEAP1, CTLA-4, 4-1BB (CD137), OX40, CD28, CD40, CD47, CD70, CD80, CD 122, GTIR, A2AR, B7-H3 (CD276), B7-H4, IDO, KIR, Tim-3, NY-ESO-1, GPC3, CLL-1, BCMA, a mucin family (MUC1, MUC2, MUC3A, MUC3B, MUC4, MUC5AC, MUC5B, MUC6, MUC7, MUC8, MUC12, MUC13, MUC15, MUC16, MUC17, MUC19, MUC20), CD19, CD20, CD22, CD30, CD33, CD52, a chemical chemokine receptor family (CCR1, CCR2, CCR3, CCR4, CCR5, CCR6, CCR7, CCR8, CCR9, CCR10, CCL27, CCL28, CX3CR1, CXCR1, CXCR2, CXCR3, CXCR4, CXCR5, CXCR6), PSMA, CEA, HDAC6, EpCAM, Mesothelin, TERT, TLR, TLR9, TLR4, CD33, GITR, Survivin CD123, TIGIT, TIM-3, CD73, fibroblast growth factor receptors (FGFR), vascular endothelial growth factors (FLT1, KDR/F1k-1, and VEGFR-3), hepatocyte growth factor receptors (HGFR), nerve growth factor receptors (NGFR), insulin-like growth factor receptors (IGFR), platelet-derived growth factor receptors (PDGFR), and hormone receptors (melanocortin 1 receptors (MC1R, MSHR).

7. (canceled)

8. The immunocytokine of claim 6, wherein the therapy-related cell surface antigen is selected from EGFR, HER2, PD-1, PD-L1, CLL-1, GPC-3, RSVF protein, CD19, CD20, CD22, CD30, CD33 or CD52.

9. The immunocytokine of claim 8, wherein the antibody targeting the therapy-related cell surface antigen is an antibody targeting EGFR, wherein the antibody has sequences of HCDR1, HCDR2 and HCDR3 contained in heavy chain as shown in SEQ ID NO: 32, and sequences of LCDR1, LCDR2 and LCDR3 contained in light chain as shown in SEQ ID NO: 34; or has a VH sequence contained in the heavy chain as shown in SEQ ID NO: 32, and a VL sequence contained in the light chain e as shown in SEQ ID NO: 34.

10. The immunocytokine of claim 8, wherein the antibody targeting the therapy-related cell surface antigen is an antibody targeting HER2, wherein the antibody has sequences of HCDR1, HCDR2 and HCDR3 contained in heavy chain as shown in SEQ ID NO: 18, and sequences of LCDR1, LCDR2 and LCDR3 contained in light chain as shown in SEQ ID NO: 20; or has a VH sequence contained in the heavy chain as shown in SEQ ID NO: 18, and a VL sequence contained in the light chain as shown in SEQ ID NO: 20.

11. The immunocytokine of claim 8, wherein the antibody targeting the therapy-related cell surface antigen is an antibody targeting PD-1, wherein the antibody has sequences of HCDR1, HCDR2 and HCDR3 contained in heavy chain as shown in SEQ ID NO: 76, and sequences of LCDR1, LCDR2 and LCDR3 contained in light chain as shown in SEQ ID NO: 78; or has a VH sequence contained in the heavy chain as shown in SEQ ID NO: 76, and a VL sequence contained in the light chain as shown in SEQ ID NO: 78.

12. The immunocytokine of claim 8, wherein the antibody targeting the therapy-related cell surface antigen is an antibody targeting PD-L1, wherein the antibody has sequences of HCDR1, HCDR2 and HCDR3 contained in heavy chain as shown in SEQ ID NO: 36, and sequences of LCDR1, LCDR2 and LCDR3 contained in light chain as shown in SEQ ID NO: 38; or has sequences HCDR1, HCDR2 and HCDR3 contained in heavy chain as shown in SEQ ID NO: 80, and sequences of LCDR1, LCDR2 and LCDR3 contained in light chain as shown in SEQ ID NO: 82; or has a VH sequence contained in the amino acid sequence as shown in SEQ ID NO: 36, and a VL sequence contained in the amino acid sequence as shown in SEQ ID NO: 38; or has a VH sequence contained in the heavy chain as shown in SEQ ID NO: 80, and a VL sequence contained in the light chain as shown in SEQ ID NO: 82.

13. The immunocytokine of claim 8, wherein the antibody targeting the therapy-related cell surface antigen is an antibody targeting an F protein of a RSV virus, wherein the antibody has sequences of HCDR1, HCDR2 and HCDR3 contained in heavy chain as shown in SEQ ID NO: 54, and sequences of LCDR1, LCDR2 and LCDR3 contained in light chain as shown in SEQ ID NO: 56; or has a VH sequence contained in the heavy chain as shown in SEQ ID NO: 54, and a VL sequence contained in the light chain as shown in SEQ ID NO: 56.

14. The immunocytokine claim 1, wherein the antibody targeting the therapy-related cell surface antigen further comprises an Fc fragment;
preferably, the Fc fragment is selected from human IgG1, IgG2, IgG3 or IgG4.

15. (canceled)

16. The immunocytokine of claim 1, wherein the linker is a cleavable linker or non-cleavable linker;
preferably, wherein the linker is independently selected from GGGGSGGGGSGGGGSG, GSPLGVRGS, GSPLGVR, PLGVR, GGGGSGPLGVRGGGGSG or GGGGSGPLGVR.

17. (canceled)

18. The immunocytokine claim 1, wherein the immunocytokine comprises a heavy chain and a light chain having the following amino acid sequences, respectively: SEQ ID NO: 2 and SEQ ID NO: 8; SEQ ID NO: 6 and SEQ ID NO: 4; SEQ ID NO: 68 and SEQ ID NO: 8; SEQ ID NO: 70 and SEQ ID NO: 4; SEQ ID NO: 10 and SEQ ID NO: 12; SEQ ID NO: 14 and SEQ ID NO: 16; SEQ ID NO: 22 and SEQ ID NO: 24; SEQ ID NO: 26 and SEQ ID NO: 28; SEQ ID NO: 40 and SEQ ID NO: 24; SEQ ID NO: 44 and SEQ ID NO: 28; SEQ ID NO: 46 and SEQ ID NO: 48; SEQ ID NO: 42 and SEQ ID NO: 58; SEQ ID NO: 84 and SEQ ID NO: 86; SEQ ID NO: 60 and SEQ ID NO: 62; SEQ ID NO: 64 and SEQ ID NO: 66; SEQ ID NO: 115 and SEQ ID NO: 116; SEQ ID NO: 117 and SEQ ID NO: 118; SEQ ID NO: 119 and SEQ ID NO: 12; SEQ ID NO: 120 and SEQ ID NO: 16; SEQ ID NO: 90 and SEQ ID NO: 34; SEQ ID NO: 92 and SEQ ID NO: 34; SEQ ID NO: 94 and SEQ ID NO: 34; SEQ ID NO: 96 and SEQ ID NO: 34; SEQ ID NO: 98 and SEQ ID NO: 20; SEQ ID NO: 100 and SEQ ID NO: 20; SEQ ID NO: 110 and SEQ ID NO: 102; SEQ ID NO: 110 and SEQ ID NO: 104; SEQ ID NO: 98 and SEQ ID NO: 102; SEQ ID NO: 98 and SEQ ID NO: 104; SEQ ID NO: 100 and SEQ ID NO: 102; SEQ ID NO: 100 and SEQ ID NO: 104; SEQ ID NO: 106 and SEQ ID NO: 20; SEQ ID NO: 110 and SEQ ID NO: 108; SEQ ID NO: 106 and SEQ ID NO: 108; SEQ ID NO: 113 and SEQ ID NO: 34; SEQ ID NO: 114 and SEQ ID NO: 34.

19. A nucleic acid, encoding the immunocytokine of claim 1.

20. A vector, comprising the nucleic acid of claim 19.

21. A host cell, comprising the vector of claim 20.

22. A pharmaceutical composition, comprising a pharmaceutically acceptable carrier or a formulation and the immunocytokine of claim 1.

23. A method for treating an inflammatory disease or a cancer of a subject in need thereof, wherein the method comprises administering the subject a therapeutically effective amount of a composition, and the composition comprises the immunocytokine of claim 1 in a pharmaceutically acceptable form.
